(19) United States
(12) Patent Application Publication
(10) Pub. No.: US 2003/0194749 A1
(43) Pub. Date: Oct. 16, 2003

(54) Wortmannin derivatives as probes of cellular proteins and processes

(76) Inventors: Thomas J. Wandless, Menlo Park, CA (US); Karlene Cimprich, Menlo Park, CA (US); Gilbert Chu, Palo Alto, CA (US); Michelle Stohlmeyer, Chicago, IL (US); Cornelia Fas, Schwaebisch Gmuend (DE)

Correspondence Address: ROPES & GRAY LLP, ONE INTERNATIONAL PLACE, BOSTON, MA, 02110-2624, US

(21) Appl. No.: 10/368,248

(22) Filed: Feb. 18, 2003

Related U.S. Application Data
(60) Provisional application No. 60/357,538, filed on Feb. 15, 2002.

(51) Int. Cl. G01N 33/53; C12N 9/12; A61K 31/366; A61K 31/353; A61K 31/7048; A61K 31/553; A61K 31/4025

(52) U.S. Cl. 435/7.1; 514/456; 514/453; 514/27; 435/194; 514/422; 514/211.08

(57) ABSTRACT

One aspect of the present invention relates to methods and reagents for profiling cells and/or subcellular environments (e.g., membrane or nuclear cellular fractions). The invention uses small molecule probes that bind covalently to protein targets, which significantly simplifies purification and identification of proteins using full length or proteolyzed proteins. Proteins, cellular components or other binding partners (collectively known as “LBP” or “lipid binding partner”) can be naturally occurring, such as proteins or fragments of proteins cloned or otherwise derived from cells, or can be artificial, e.g., polypeptides which are selected from random or semi-random polypeptide libraries.

### RELATED APPLICATION

This application claims the benefit of the filing date of U.S. Provisional Application No. 60/357,538, filed Feb. 15, 2002 and entitled “Wortmannin Derivatives as Probes of Cellular Proteins and Processes,” by Thomas Wandless and Karlene Cimprich. The entire teachings of the referenced provisional application are incorporated herein by reference.

Throughout this application, various publications are referenced by author name and publication date. Full citations for those publications may be found at the end of the specification immediately proceeding the claims. The disclosure of all referenced publications is hereby incorporated by reference into this application to describe more fully the art to which this invention pertains.

### FUNDING

The invention described herein was supported, in whole or in part, by Grant No. CA-77317 from the National Institute of Health. The Government has certain rights in the invention.

### BACKGROUND OF THE INVENTION

A large part of the signal transduction in checkpoint pathways that ensure genomic integrity is fulfilled by members of the PIK-related kinases. The PIK-related kinases belong to a superfamily of kinases (PI 3-kinase superfamily) that includes phosphoinositide 3-kinases (PI 3-kinases) as well as the phosphoinositide 4-kinases (PI 4-kinases). The PIK-related kinases share a conserved kinase domain of around 300 amino acids at their C-termini. This domain is related to the kinase domain of PI 3-kinases and includes the ATP and substrate binding region (Bosotti et al., 2000). In contrast to the PI 3-kinases however, which primarily phosphorylate phosphoinositol phosphates (PIPs), PIK-related kinases are protein kinases with a preference for serine or threonine residues. They do not appear to be capable of phosphorylating lipids (Stein et al., 2000). In addition to the sequence homology in the kinase domain, family members exhibit a similar overall structural organization as shown in FIG. 1. Members of the PIK-related kinases are high molecular weight enzymes (from about 300 kD to >500 kD) that share little homology at the N-terminus. At the C-terminus, however, flanking the kinase domain, two further regions of homology have been defined: the FAT (FRAP, ATM and TRRAP) domain and a domain at the extreme carboxy-terminus known as the FATC (carboxyterminal counterpart of FAT) domain (Bosotti et al., 2000). The functions of these domains are not known.

Members of the PIK-related kinase family have been shown to be essential in a variety of processes including cell cycle progression, cell cycle checkpoints, chromosome maintenance, DNA repair and V(D)J recombination (Keith et al., 1995). In humans, members of the PIK-related kinase family include ATM (ataxia telangiectasia mutated), ATR (ATM and Rad3 related, also called FRP1) (Cimprich et al., 1996; Bentley et al., 1996), DNA-PKcs (catalytic subunit of DNA dependant protein kinase) (Hartley et al., 1995), FRAP (FKBP12-rapamycin-associated protein, also called mTOR, RAFT, RAPT) (Brown et al., 1994; Sabers et al., 1994; Chiu et al., 1994) and the newly characterized SMG1 (suppressor with morphogenic defect of genetalia) (Denning et al., 2001). Another potential member of this family is TRRAP (transactivation/transformation-domain associated protein) which shows homology to ATM but which lacks several of the typically conserved catalytic residues found in the kinase domain of all PIK-related kinases (Grant et al., 1998). Consistent with the lack of catalytic residues, TRRAP has not been shown to possess kinase activity (McMahon et al., 1998). ATM and ATR seem to be key proteins in the transduction of damage signals, and they may also be involved in sensing different kinds of DNA damage. DNA-PK plays an important role in the detection and repair of double-strand breaks while FRAP is involved in nutrient sensing and regulation of the G1/S transition (Kuruvilla et al., 1999). SMG1 appears to be involved in nonsense-mediated mRNA decay according to the function of the Caenorhabditis elegans analog (Denning et al., 2001). Recent evidence is evolving which indicates that SMG1 may join ATR and ATM as a stress-response protein kinase (Abraham, 2001). TRRAP's possible function lies in acting as a molecular scaffold during gene transcription (Park et al., 2001).

Disruption of DNA-PK function in mice leads to radiosensitivity and immunodeficency. DNA-PK has shown to play a role in V(D)J recombination, the process by which the diversity of the T-cell receptor and antibodies are generated (Bosma et al., 1991). Further, it is known to function in the detection and repair of DNA double-strand breaks.

DNA-PK is composed of a 470 kD catalytic subunit DNA-PK_{cs} and a targeting heterodimer Ku70-Ku80 (Hartley et al., 1995). The catalytic subunit exhibits an intrinsic DNA binding activity that can greatly be increased by the Ku heterodimer, and its catalytic serine/threonine kinase activity is highly increased by the presence of DNA (Gottlieb et al., 1993). Many in vitro substrates of DNA-PK, which include a variety of transcription factors, repair proteins and chromatin components, have been identified (Anderson et al., 1992), but further verification of their in vivo relevance has proven difficult. After some controversial discussion, most recent evidence suggests that DNA-PK does not act to signal cell cycle arrest through p53 (Smith et al., 1999b; Lees-Miller et al., 1992). There is some evidence that DNA-PK may be involved in signaling damage to the apoptosis machinery. However, the primary function for DNA-PK appears to lie in the detection and repair of double-strand breaks by a mechanism called non-homologous end joining repair (NHEJ) (Featherstone et al., 1999). By this repair mechanism, DNA ends are put together without a sister chromatid template. The current model is that DNA double strand breaks are recognized by the Ku subunits. DNA-PK_{cs} is then recruited to the site and assembled into an active DNA-PK complex that mediates synapsis between the opposing ends. Now, DNA-PKcs phosphorylates Ku and DNA-PK_{cs} bound to opposing ends which leads to dissociation of the complex and inactivation of DNA-PK. Later stages of the repair process involve a second complex containing RAD50, MRE11 and NBS1 and a third complex containing DNA ligase-IV-XRCC4 which rejoins the ends (Kanaar et al., 1998).

ATM is the gene mutated in the autosomal recessive disorder ataxia telangiectasia, and patients with this disease are hypersensitive to ionization radiation, suffer from extensive neurodegeneration and have a predisposition to cancer (Meyn, 1995). Studies of these patients, mice in which ATM was disrupted (Barlow et al., 1996) and cells from both patients and mice revealed that ATM is mainly involved in the response to double-strand breaks which are frequently caused by ionization radiation (IR) and is a key player in G1/S, S and G2/M checkpoints (Durocher et al., 2001). The kinase activity of the protein ATM towards p53, an in vivo substrate, is stimulated by binding to DNA. But in contrast to DNA-PK, experiments indicate that ATM can directly bind to DNA, with a preference for DNA ends (Smith et al., 1999a; Suzuki et al., 1999). Gel filtration studies have provided evidence which suggests that ATM is associated with protein complexes of high molecular weight (>2 MD) (Shiloh, 2001). The identification of the components of this complex is being actively pursued in the hope of gaining new insights into both the upstream activators and downstream targets of ATM. Recently, a fraction of cellular ATM was found in a large protein complex BASC (BRCA1-associated genome surveillance complex), that was immunoprecipitated using an anti-BRCA1 antibody. This complex was also shown to contain a number of repair proteins (Wang et al., 2000) and chromatin remodeling factors (Bochar et al., 2000).

Currently, no genetic disorder has been linked to mutations in the ATR gene, likely resulting from the essential nature of its function. Disruption of ATR in mice leads to lethality only a few days after implantation and studies indicate that cells in ATR−/− blastocytes undergo extensive chromosomal fragmentation (Brown et al., 2000; de Klein et al., 2000). Most insights into ATR function have been gained with a cell line expressing a kinase inactive form of ATR that acts as a dominant negative (Cliby et al., 1998). These studies suggest that ATR is involved in the detection and transduction of different damage signals caused by UV, hydroxyurea (HU), alkylating agents and ionizing radiation (IR), and that it acts at the G1/S, S and G2/M damage checkpoints (Durocher et al., 2001). ATR substrates by which the signaling cascades are mediated include p53, Chk1 and BRCA1 (Tibbetts at al., 1999; Tibbetts et al., 2000; Liu et al., 2000).

ATR is a 301 kD protein that is known to be in a large molecular weight complex (>2 MD) in the cell (Wright et al., 1998) although little about its associated proteins is known. By coimmunoprecipitation experiments with an ATR antibody, two components of the nucleosome remodeling and deacetylation complex (HDAC2 and CHD4) were identified as ATR-associated proteins (Schmidt et al., 1999). Another ATR related protein, ATRIP, has been recently identified (Cortez et al., 2001). Another question that may be related to ATR's ability to bind DNA involves the mechanism by which the damage checkpoint gets activated. The identification of proteins that act as sensors of DNA damage is an active area of research.

Several compounds have been discovered that can inhibit members of the PI 3-kinase superfamily. Wortmannin is a steroid derivate that was originally isolated from the soil bacteria Penicillium wortmannii and is a fungal toxin (Brian et al., 1957). Demethoxyviridin is much less studied than wortmannin but closely related to that compound. Although it is slightly more potent than wortmannin, it is significantly less stable (Woscholski et al., 1994). LY294002 is a morpholino derivative of the broad spectrum kinase inhibitor quercetin (Vlahos et al., 1994).

LY294002 is a synthetic compound that was designed as a PI 3-kinase inhibitor (Vlahos et al., 1994). It is a competitive, reversible inhibitor which acts at the ATP-binding site (Walker et al., 2000). Although the IC₅₀ of LY294002 for p110α is 1.4 μM (Vlahos et al., 1994), about 500 times higher than the IC₅₀ of wortmannin, this compound is frequently used as a specific inhibitor in cell biology experiments since it is much more stable in solution than wortmannin (Walker et al., 2000). It has been shown to be able to inhibit DNA-PK with a Kᵢ of 6.0 μM (Izzard et al., 1999) and mTOR with an IC₅₀ of about 3 μM (Brunn et al., 1996). LY 294002 was screened against a broad range of unrelated kinases and it was shown to inhibit casein kinase 2 at concentrations on the order of those that inhibit PI 3-kinase (Davies et al., 2000).

Wortmannin is unstable in aqueous solution, undergoing hydrolysis of the furan ring, a reaction that destroys its inhibitory effect (Baggiolini et al., 1987). Due to its instability and toxicity, its pharmaceutical potential is rather low (Stein et al., 2000). However, the compound has been widely used to study processes mediated by PI3K and PIK-related kinases. The mechanism, by which wortmannin acts has been primarily studied in the context of PI3K. By site-directed mutagenesis, it was found that wortmannin reacts with lysine 802 in the ATP-binding site of the catalytic center (Wymann et al., 1996). This was further verified by solving the crystal structure of a porcine PI3Kγ C-terminal fragment bound to wortmannin in the ATP binding pocket. Wortmannin fills the active site of porcine PI3Kγ thereby inducing a conformational change in the catalytic domain (Walker et al., 2000). Structure-activity studies have shown that C21 of wortmannin is essential to its function (Norman et al., 1996), leading to the following mechanism of action presented in FIG. 2; studies suggest that the ε-amino group of Lys-802 nucleophilically attacks C21 of wortmannin, leading to the opening of the furan ring and the formation of an enamine. Thus the kinase becomes covalently attached to the drug. The enamine is in equilibrium with a Schiff base, which is relatively stable under physiologically conditions but is easily hydrolyzed under acidic conditions. More stability can be achieved by reducing the Schiff base to its imine form with NaCNBH₃ (Wymann et al., 1996).

Wortmannin has been shown to be an irreversible, potent and specific inhibitor of PI 3-kinases at low nanomolar concentrations (IC₅₀, 2-4.2 nM) (Arcaro et al., 1993; Powis et al., 1994; Ui et al., 1995), although it does not show selectivity among most isoforms of the PI 3-kinases (Stein et al., 2000). At higher concentrations, wortmannin has also been shown to react with members of the PIK-related family which is of particular interest. So far, DNA-PK is the most sensitive member of the PIK-related kinases with an IC₅₀ of 16 nM (Sarkaria et al., 1998), followed by ATM (IC₅₀, 150 nM) (Sarkaria et al., 1998), FRAP (IC₅₀, 0.1-1 μM) (Brunn et al., 1996) and ATR (IC₅₀, 1.8 μM) (Sarkaria et al., 1998).

Besides the above kinases, myosin light chain kinase, PIP₂-phospholipase C, phospholipase D and phospholipase A2 have been shown to be inhibited by wortmannin. Although, except for phospholipase A2, higher wortmannin concentrations than for PI 3-kinases are needed to reach inhibition (Cross et al., 1995).

### SUMMARY OF THE INVENTION

Although the poor selectivity wortmannin shows in its ability to inhibit members of the PI 3-kinase superfamily and the higher concentrations (μM) that are required to inhibit members of the PIK-related kinases can cause difficulties in targeting a specific family member, these properties can also prove to be powerful advantages in targeting a whole enzyme family. Proteomic based approaches to target and profile enzyme families have been successfully explored by using radiolabeled vinyl sulfones as selective reagent for members of the proteasome family proteases (Bogyo et al., 1997), by developing epoxide electrophiles as activity-dependent tool for cysteine proteases (Greenbaum et al., 2000) or by using biotinylated fluorophosphonate to profile serine hydrolases (Liu et al., 1999; Kidd et al., 2001).

This invention demonstrates the utility of isolating and characterizing proteins binding to Wotmannin or its analog, or lipid kinase inhibitors in general. The invention also specifically contemplates broad applicability for the isolation of binding partners for other lipid kinases.

One aspect of the invention provides a method for identifying binding partners which bind to a wortmannin moiety comprising:

a. providing a wortmannin bait moiety including wortmannin or an analog thereof been derivatized to a solid support or molecular or chemical tag for purifying or identifying the bait moiety;

b. contacting the bait moiety with a library of binding partners and isolating from the library binding partners, if any, which bind to the bait moiety;

c. identifying those members of the binding partner library which specifically bind to the bait moiety.

Another aspect of the invention provides a method for identifying kinases comprising:

a. providing a lipid kinase inhibitor bait moiety being derivatized to a solid support or molecular or chemical tag for purifying or identifying the bait moiety, which lipid kinase inhibitor forms a covalent adduct with lipid kinases and has a Ki for inhibition of a lipid kinase of 50 μM or less;

b. contacting the bait moiety with a library of binding partners and isolating from the library binding partners, if any, which bind to the bait moiety;

c. identifying those members of the binding partner library which specifically bind to the bait moiety.

In preferred embodiments, the bait moiety is a covalent inhibitor of a phosphatidylinositol kinase. In certain preferred embodiments, the lipid kinase inhibitor is selected from the group consisting of wortmannin, hydroxywortmannin, LY294002, demethoxyviridin, quercetin, myricetin and staurosporine, and analogs thereof. In certain preferred embodiments, the lipid kinase inhibitor is derivatized to the solid support or molecular or chemical tag through a cross-linking moiety which is covalently attached to C11 the wortmannin or wortmannin analog. In certain preferred embodiments, fluorescein and other fluorescent moieties may be used as molecular or chemical tags.

Another aspect of the invention provides a method wherein the bait moiety is represented in the general formula

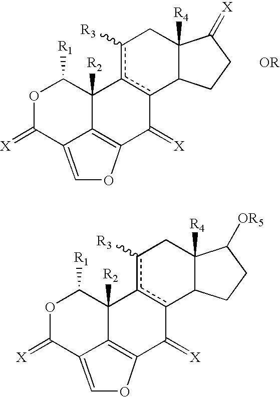

wherein

X, independently for each occurrence, represents O or S,

R₁ represents —(CH₂)ₙ—X—R₅,

R₂, R₄ and R₅, independently, represent H or a C1-C6 alkyl,

R₃ represents S-L-,

S represents a solid support or molecular or chemical tag for purifying or identifying the bait moiety,

L represents a linker, and

n is 0, 1, 2 or 3.

In certain preferred embodiments, the library of binding partners is a polypeptide library, e.g., including at least 10 different polypeptides, more preferably at least 100, 1000, or even 10,000 different proteins. For instance, the polypeptide library can be an expression library, such as derived from replicable genetic display packages. In other embodiments, the library of binding partners is a synthetic polypeptide library. In other embodiments, the library of binding partners is a library of cellular components. In other embodiment, the library of binding partners is a cell lysate, nuclear extract, or partially purified protein preparation.

The identity of those members of the binding partner library which specifically bind to the lipid kinase inhibitor bait moiety can be determined by mass spectroscopy.

The members of the binding partner library which specifically bind to the lipid inhibitor bait moiety can be separated and/or identified by SDS-PAGE.

Another aspect of the present invention provides a screening assay comprising:

a. providing a reaction mixture including a binding partner identified as described above identified by binding to a wortmannin moiety or a lipid kinase inhibitor moiety;

b. contacting the binding partner with a test compound;

c. determining if the test compound specifically binds to the binding partner.

In preferred embodiments, the assay is repeated for a variegated library of at least 100 different test compounds, even more preferably at least 100, 1000 or even 10,000 different test compounds. Exemplary test compounds which can be screened for activity in the subject assays include peptides, nucleic acids, carbohydrates, small organic molecules, and natural product extract libraries, such as isolated from animals, plants, fungus and/or microbes. In preferred embodiments, the test compound which is identified as able to bind to the binding partner is further tested for the ability to inhibit or activate one or more cellular kinases.

In certain preferred embodiments, the reaction mixture is a whole cell. In other embodiments, the reaction mixture is a cell lysate, nuclear extract, or purified protein composition.

In certain embodiments, a test compound which is identified as able to bind to the binding partner is further tested for the ability to inhibit or mimic the activity of a lipid kinase inhibitor moiety.

Still another aspect of the present invention provides a method of conducting a drug discovery business comprising:

a. providing a wortmannin bait moiety being derivatized to a solid support or molecular or chemical tag for purifying or identifying the bait moiety;

b. contacting the wortmannin bait moiety with a library of binding partners;

c. identifying those members of the binding partner library which specifically bind to the wortmannin bait moiety;

d. providing a reaction mixture including a binding partner identified in step (c) as able to specifically bind to the wortmannin bait moiety;

e. contacting the binding partner with a test compound;

f. determining if the test compound specifically binds to the binding partner;

g. formulating a pharmaceutical preparation including one or more compounds identified in step (f) as able to inhibit or mimic the activity of a wortmannin moiety.

Yet another aspect of the invention provides a method of conducting a drug discovery business comprising:

a. identifying those members of a binding partner library which specifically bind to a wortmannin bait moiety;

b. identifying compounds by their ability to agonize or antagonize a binding partner identified in step (a);

c. conducting therapeutic profiling of a compound identified in step (b), or further analogs thereof, for efficacy and toxicity in animals;

d. formulating a pharmaceutical preparation including one or more agents identified in step (iii) as having an acceptable therapeutic profile.

In preferred embodiments the method will include an additional step of establishing a distribution system for distributing the pharmaceutical preparation for sale, and/or establishing a sales group for marketing the pharmaceutical preparation.

Yet another aspect of the invention provides a method of conducting a target discovery business comprising:

a. providing a wortmannin bait moiety being derivatized to a solid support or molecular or chemical tag for purifying or identifying the bait moiety;

b. contacting the wortmannin bait moiety with a library of binding partners;

c. identifying those members of the binding partner library which specifically bind to the wortmannin bait moiety;

d. licensing, to a third party, the rights for drug development for a binding partner identified in step (c) as able to specifically bind to the wortmannin bait moiety.

Another aspect of the invention provides a method of generating compounds, which are able to bind to the binding partner identified by specifically binding to a wortmannin bait moiety, for wortmannin-binding proteins, comprising:

a. providing a wortmannin bait moiety being derivatized to a solid support or molecular or chemical tag for purifying or identifying the bait moiety;

b. contacting the wortmannin bait moiety with a library of binding partners;

c. identifying those members of the binding partner library which specifically bind to the wortmannin bait moiety;

d. providing a reaction mixture including a binding partner identified in step (c) as able to specifically bind to the wortmannin bait moiety;

e. contacting the binding partner with a test compound;

f. determining if the test compound binds to the binding partner;

g. formulating a pharmaceutical preparation including one or more compounds identified in step (f) as able to inhibit or mimic the activity of a wortmannin moiety.

One aspect of the invention provides a composition including a bait moiety represented in the general formula

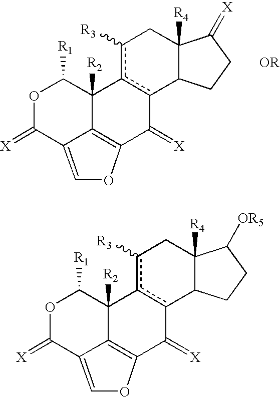

wherein

X, independently for each occurrence, represents O or S,

R₁ represents —(CH₂)ₙ—X—R₅,

R₂, R₄ and R₅, independently, represent H or a C₁-C₆ alkyl,

R₃ represents S-L-,

S represents a solid support or molecular or chemical tag for purifying or identifying the bait moiety,

L represents a linker, and

n is 0, 1, 2 or 3.

One aspect of the invention provides a method for profiling wortmannin-binding components of a cellular lysate, comprising:

a. providing a wortmannin bait moiety including wortmannin or an analog thereof being derivatized to a solid support or molecular or chemical tag for purifying the bait moiety;

b. contacting the bait moiety with a cell lysate and isolating from the lysate binding partners, if any, which bind to the bait moiety;

c. identifying those binding partners which specifically bind to the bait moiety.

The identity of the binding partners which specifically bind to the bait moiety can be determined by mass spectroscopy.

Another aspect of the invention provides a method for profiling wortmannin-binding components of a cell, comprising:

a. providing a wortmannin bait moiety including wortmannin or an analog thereof being derivatized to a molecular or chemical tag for visualizing the bait moiety in a cell;

b. contacting the bait moiety with a cell and determining the cellular localization(s) of the bait moiety.

Another aspect of the invention provides a method for profiling wortmannin-binding components, comprising:

a. providing a wortmannin bait moiety including wortmannin or an analog thereof being derivatized to a molecular or chemical tag for visualizing the bait moiety in a cell;

b. contacting the bait moiety with a library of binding partners;

c. separating the components using SDS-PAGE and determining the location of the bait moiety on the gel. Another aspect of the invention provides a method for profiling wortmannin-binding components comprising:

a. providing a wortmannin bait moiety including wortmannin or an analog thereof being derivatized to a molecular or chemical tag for purifying or identifying the bait moiety;

b. contacting the bait moiety with a library of cellular components, wherein the library of cellular components has been derived from cells that have been exposed to a first set of conditions, and identifying those members of the cellular component library which specifically bind to the bait moiety;

c. contacting the bait moiety with a library of cellular components, wherein the library of cellular components has been derived from cells that have been exposed to a second set of conditions, and identifying those members of the cellular component library which specifically bind to the bait moiety;

d. comparing the cellular components identified in (b), with the cellular components identified in (c), wherein a difference between the cell components identified in (b) and the cellular components identified in the (c) indicates that the difference in the conditions cause a change in the cellular component that can be used to profile the binding components.

Another aspect of the invention provides a method for identifying the phosphorylation state of wortmannin binding partners comprising:

a. providing a wortmannin bait moiety including wortmannin or an analog thereof being derivatized to a molecular or chemical tag for purifying or identifying the bait moiety;

b. contacting the bait moiety with a library of cellular components that have been derived from cells that have been exposed to a first set of conditions, and identifying those members of the cellular component library which specifically bind to the bait moiety;

c. contacting the bait moiety with a library of cellular components that have been derived from cells that have been exposed to a second set of conditions, and identifying those members of the cellular component library which specifically bind to the bait moiety;

d. comparing the cellular components identified in (b), with the cellular components identified in (c), thereby identifying cellular components that differently phosphorylated between the first and second set of conditions.

In one embodiment, the first set of conditions is the presence of phosphatase inhibitors, and the second set of conditions is the absence of phosphatase inhibitors. In another embodiment, the first set of conditions is the presence of one or more growth factors, and the second set of conditions is the absence of said one or more growth factors.

Another aspect of the invention provides a method for identifying the phosphorylation state of wortmannin binding partners comprising:

a. providing a wortmannin bait moiety including wortmannin or an analog thereof being derivatized to a molecular or chemical tag for purifying or identifying the bait moiety;

b. contacting the bait moiety with a library of cellular components that have been derived from differentiated cells, and identifying those members of the cellular component library which specifically bind to the bait moiety;

c. contacting the bait moiety with a library of cellular components that have been derived from undifferentiated cells, and identifying those members of the cellular component library which specifically bind to the bait moiety;

d. comparing the cellular components identified in (b), with the cellular components identified in (c), thereby identifying cellular components that are differently phosphorylated between differentiated and undifferentiated cells.

Another aspect of the invention provides a method for identifying the phosphorylation state of wortmannin binding partners comprising:

a. providing a wortmannin bait moiety including wortmannin or an analog thereof being derivatized to a molecular or chemical tag for purifying or identifying the bait moiety;

b. contacting the bait moiety with a library of cellular components that have been derived from cancer cells, and identifying those members of the cellular component library which specifically bind to the bait moiety;

c. contacting the bait moiety with a library of cellular components that have been derived from non-cancer cells, and identifying those members of the cellular component library which specifically bind to the bait moiety;

d. comparing the cellular components identified in (b), with the cellular components identified in (c), thereby identifying cellular components that are differently phosphorylated between cancer and non-cancer cells.

In one embodiment, the members of the cellular components that specifically bind to the bait moiety are identified by SDS-PAGE. In another embodiment, the members of the cellular components that specifically bind to the bait moiety are identified by two-dimensional gel electrophoresis. In another embodiment, the bands in the SDS-PAGE that correspond to the cellular components identified in step (b) and (c) are identified.

In one embodiment, the members of the cellular components that specifically bind to the bait moiety are affinity enriched. In another embodiment, the members of the cellular components that specifically bind the bait moiety are identified using mass spectroscopy.

### BRIEF DESCRIPTION OF THE DRAWINGS

FIG. 1. Typical structural features of PIK-related kinases. A very long and nonhomologous N-terminus, conserved FAT (FRAP, ATM and TRRAP), FATC (C-terminal counterpart of FAT) and PI3K kinase domains are characteristics of PIK-related kinase family members. TRRAP is the only exception, missing some of the typically conserved residues in the kinase domain.

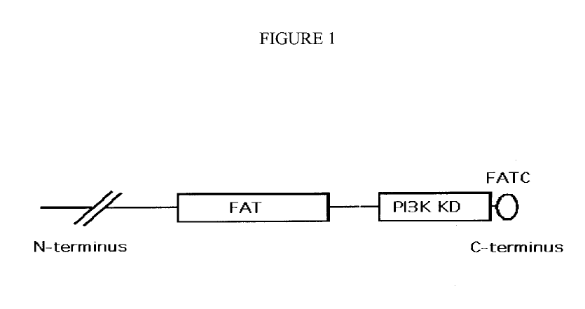

FIG. 2. Proposed mechanism of PI 3-kinase inhibition by wortmannin (Norman et al., 1996). PI 3-kinase inhibition occurs by nucleophilic addition of a lysine in the kinase to the electrophilic C21 position of wortmannin.

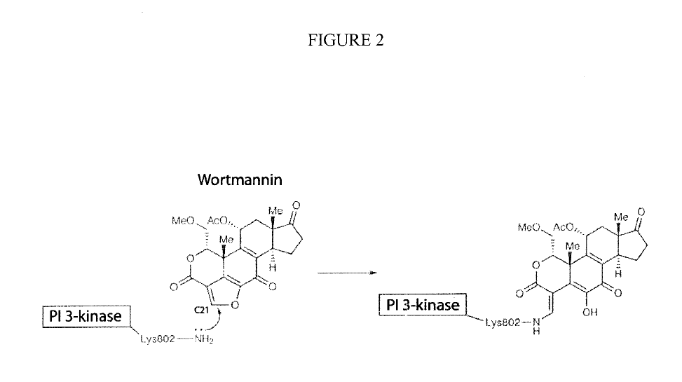

FIG. 3. Western blot showing the labeling of proteins with biotin-wortmannin in cell lysates prepared in the presence or absence of phosphatase inhibitors. The arrows denote proteins with apparent differential reactivity to biotin-wortmannin. Approximate molecular weights are indicated on the left.

### DETAILED DESCRIPTION OF THE INVENTION

A) Overview

Members of the PI 3-kinase superfamily play roles in important cellular processes such as signal transduction, cell cycle regulation and DNA repair (Keith et al., 1995), (Stein et al., 2000). The family of the PIK-related kinases is a subgroup of this family whose members function in cell cycle regulation, chromosome maintenance, DNA repair, V(D)J recombination and DNA repair (Keith et al., 1994). Besides dimethoxyviridin, wortmannin is the only known inhibitor of PIK-related kinases that can form a covalent bond. This is an essential prerequisite for the bait moieties of the invention.

With wortmannin derivatives, it is possible to specifically target members of the PI 3-kinase superfamily as a whole, or in groups, by varying parameters such as inhibitor concentration.

In one embodiment, the wortmannin is linked through a water soluble polyethylene glycol (“PEG”) linker to biotin. The linkage of wortmannin to biotin allows one to take advantage of detection and purification assays that are based on the high affinity interaction between biotin and streptavidin/avidin and could lead to the identification of interacting proteins and/or further characterization of these proteins.

In another embodiment, the wortmannin is linked to a fluorescent label. The linkage of wortmannin to a fluorescent label allows one to take advantage of detection assays that are based on the detection of the fluorescence, and could lead to the identification of interacting proteins and/or further characterization of these proteins.

In another embodiment, the wortmannin is linked to biotin and to a fluorescent label. In one embodiment, the wortmannin is liked to biotin and to a fluoresecent label via a linker.

In another embodiment, the wortmannin is linked to biotin and to a lipid. In one embodiment, the lipid is a cationic lipid. In one embodiment the wortmannin is linked to biotin and to a lipid via a linker. In one embodiment, the linker binds to all three components: wortmannin, biotin and lipid. In another embodiment, the linker only binds to two components, and as a result the three components are bound sequentially.

One aspect of the present invention relates to methods and reagents for identifying proteins or other cellular components (collectively “LBP” or “Lipid kinase inhibitor Binding Partner”). The LBPs can be naturally occurring, such as proteins or fragments of proteins cloned or otherwise derived from cells, or can be artificial, e.g., polypeptides which are selected from random or semi-random polypeptide libraries.

In one embodiment, the method of the present invention comprises providing a lipid kinase inhibitor moiety which includes a “sequestration tag”, and contacting the lipid kinase inhibitor with a structurally diverse (variegated) library of binding partners (such as polypeptides, cellular components or other molecules) under conditions wherein binding of lipid kinase inhibitors to library molecules can occur such that the resulting complexes are enriched for library molecules which specifically (as opposed to non-specifically) bind the lipid kinase inhibitors. Library molecules which specifically bind to the lipid kinase inhibitors are isolated from the library, or their identity is otherwise determined, e.g., by the presence of a tag associated with the LBP which is a unique identifier of the LBP. The library of binding partners may be a polypeptide library. The polypeptide library can be provided as part of a replicable genetic display package, an expression library (especially an intracellular expression library), a synthetic polypeptide library or other form.

In other embodiments, the system can be reversed and a polypeptide can be used to screen a library of structurally diverse lipid kinase inhibitors to identify lipid kinase inhibitors which selectively bind to the polypeptide.

In one embodiment, the method of the present invention comprises providing a lipid kinase inhibitor moiety which includes a molecular or chemical tag for identifying the bait moiety, and contacting the lipid kinase inhibitor moiety with a structurally diverse (variegated) library of binding partners (such as polypeptides, cellular components or other molecules) under conditions wherein binding of the lipid kinase inhibitor moiety to the library molecules can occur, and identifying the library components that specifically bind the lipid kinase inhibitor moiety. In one embodiment, the lipid kinase inhibitor moiety is fluorescently labeled. The binding partners which specifically bind to the fluorescently labeled lipid kinase inhibitor moiety may be identified using gel electrophoresis, preferably two-dimensional gel electrophoresis.

Lipid kinase inhibitor moieties may bind differently to binding partners that are modified by disease states or by altered environmental conditions. Therefore, lipid kinase inhibitor moieties may be used to monitor changes in the binding partners in disease states, or under altered environmental conditions. In one embodiment the disease state is cancer. In one embodiment the altered environmental condition is the presence of phosphate, or phosphatase inhibitors. Example 3 indicates that the phosphorylation state of a binding partner may affect the biding to the wortmannin moiety. This characteristic of the lipid kinase inhibitor moieties can be used to determine the phosphorylation state of the LPBs. Further, this characteristic of the lipid kinase inhibitor moieties can be use to profile the LPBs in two different cells or tissues hat have been subject to different conditions.

Further, the wortmannin lipid kinase inhibitor bait moieties can be used in drug screening to identify drugs that interact with the binding partners that bind to the lipid kinase inhibitor bait moieties.

Another aspect of the present invention relates to the LBPs which are identified by the subject method. Such molecules can be used as drug screening targets, e.g., for drugs which alter the activity of the LBP (such as its ability to bind a lipid kinase inhibitor) or which alter the level of the LBP in the cell. Moreover, the level of an LBP in a cell can be determined for diagnostic or prognostic purposes.

Where the LBP is a protein, the invention also relates to nucleic acids which encode the protein or a fragment thereof. The invention also contemplates nucleic acids which hybridize to the coding sequence for an LBP, e.g., which may be useful as amplimers, probes, primers or antisense.

Another aspect of the present invention relates to antibodies, e.g., monocolonal, purified and/or recombinant, which are immunoselective for an LBP.

Still another aspect of the present invention relates to drug screening assays for identifying compounds, e.g., such as small organic molecules (MW<1000 amu) which inhibit or potentiate the activity of an LBP. For instance, the assay can be used to identify compounds which inhibit or potentiate an intrinsic enzymatic activity of an LBP, or the ability of the LBP to bind to other molecules, e.g., to lipid kinase inhibitors, to proteins, to nucleic acids.

Yet another aspect of the present invention relates to the use the LBPs, or compounds which agonize or antagonize, as the case may be, the activity of an LBP, for the treatment or prevention of a disorder or unwanted effect mediated by a lipid kinase inhibitor.

B) Definitions

Before further description of the invention, certain terms employed in the specification, examples and appended claims are, for convenience, collected here.

The term “LBP,” “Lipid kinase inhibitor Binding Partner” or “binding partner” generally means a binding partner for at least one inhibitor of a PI3K superfamily kinase. The binding partner is not limited to polypeptides, but can be anything (lipids, nucleic acids, small molecules, etc.) that can potentially bind such inhibitors. “Lipid kinase” here refer to the PI3K superfamily of kinases. Some of the members belonging to this superfamily may possess lipid kinase activity, while others may be protein kinases, yet others may contain both activities. Therefore, the term “lipid kinase inhibitor” is not limited to inhibitors of those superfamily members that are lipid kinases, but rather, it is meant to be an inhibitor of any family members regardless of their kinase activity or specificity. Examples of such lipid kinase inhibitor are Wortmannin and LY294002.

“Cellular component” means anything that originates from a cell. Thus, it is not limited to polypeptides. For example, it can be small molecules, nucleic acids, lipids, polysaccharides, etc.

“Fatty acids” are long-chain hydrocarbon molecules containing a carboxylic acid moiety at one end. The numbering of carbons in fatty acids begins with the carbon of the carboxylate group. Fatty acids that contain no carbon-carbon double bonds are termed saturated fatty acids; those that contain double bonds are unsaturated fatty acids. The numeric designations used for fatty acids come from the number of carbon atoms, followed by the number of sites of unsaturation (eg, palmitic acid is a 16-carbon fatty acid with no unsaturation and is designated by 16:0). The site of unsaturation in a fatty acid is indicated by the symbol Δ and the number of the first carbon of the double bond (e.g. palmitoleic acid is a 16-carbon fatty acid with one site of unsaturation between carbons 9 and 10, and is designated by 16:1^{Δ9}).

“Triacylglycerides” are composed of a glycerol backbone to which 3 fatty acids are esterified.

The basic structure of “phospolipids” is very similar to that of the triacylglycerides except that C-3 (sn3) of the glycerol backbone is esterified to phosphoric acid. The building block of the phospholipids is phosphatidic acid which results when the X substitution in the basic structure shown in the structure below is a hydrogen atom. Substitutions include ethanolamine (phosphatidylethanolamine), choline (phosphatidylcholine, also called lecithins), serine (phosphatidylserine), glycerol (phosphatidylglycerol), myo-inositol (phosphatidylinositol, these compounds can have a variety in the numbers of inositol alcohols that are phosphorylated generating polyphosphatidylinositols), and phosphatidylglycerol (diphosphatidylglycerol more commonly known as cardiolipins).

The term “simultaneously expressing” refers to the expression of a representative population of a polypeptide library, e.g., at least 50 percent, more preferably 75, 80, 85, 90, 95 or 98 percent of all the different polypeptide sequences of a library.

The term “random polypeptide library” refers to a set of random or semi-random polypeptides.

The language “replicable genetic display package” or “display package” describes a biological particle which has genetic information providing the particle with the ability to replicate. The package can display a fusion protein including a polypeptide derived from the variegated polypeptide library. The test polypeptide portion of the fusion protein is presented by the display package in a context which permits the polypeptide to bind to a lipid kinase inhibitor that is contacted with the display package. The display package will generally be derived from a system that allows the sampling of very large variegated polypeptide libraries. The display package can be, for example, derived from vegetative bacterial cells, bacterial spores, and bacterial viruses.

The language “differential binding means”, as well as “affinity selection” and “affinity enrichment”, refer to the separation of members of the polypeptide display library based on the differing abilities of polypeptides on the surface of each of the display packages of the library to bind to the lipid kinase inhibitor. The differential binding of a lipid kinase inhibitor by test polypeptides of the display can be used in the affinity separation of those polypeptides which specifically bind the lipid kinase inhibitor from those which do not. For example, the affinity selection protocol can also include a pre- or post-enrichment step wherein display packages capable of binding “background lipid kinase inhibitors”, e.g., as a negative selection, are removed from the library. Examples of affinity selection means include affinity chromatography, immunoprecipitation, fluorescence activated cell sorting, agglutination, and plaque lifts. As described below, the affinity chromatography includes bio-panning techniques using either purified, immobilized lipid kinase inhibitor proteins or the like, as well as whole cells.

The phrases “selective manner”, “selective binding” and “specifically bind”, with respect to binding of a test polypeptide with a lipid kinase inhibitor, refers to the binding of a polypeptide to a certain protein lipid kinase inhibitor which binding is specific for, and dependent on, the molecular identity of the protein or lipid kinase inhibitor.

The term “solid support” refers to a material having a rigid or semi-rigid surface. Such materials will preferably take the form of small beads, pellets, disks, chips, dishes, multi-well plates, wafers or the like, although other forms may be used. In some embodiments, at least one surface of the substrate will be substantially flat. The term “surface” refers to any generally two-dimensional structure on a solid substrate and may have steps, ridges, kinks, terraces, and the like without ceasing to be a surface.

The language “fusion protein” and “chimeric protein” are art-recognized terms which are used interchangeably herein, and include contiguous polypeptides comprising a first polypeptide covalently linked via an amide bond to one or more amino acid sequences which define polypeptide domains that are foreign to and not substantially homologous with any domain of the first polypeptide. One portion of the fusion protein comprises a test polypeptide, e.g., which can be random or semi-random. A second polypeptide portion of the fusion protein is typically derived from an outer surface protein or display anchor protein which directs the “display package” (as hereafter defined) to associate the test polypeptide with its outer surface. As described below, where the display package is a phage, this anchor protein can be derived from a surface protein native to the genetic package, such as a viral coat protein. Where the fusion protein comprises a viral coat protein and a test polypeptide, it will be referred to as a “polypeptide fusion coat protein”. The fusion protein further comprises a signal sequence, which is a short length of amino acid sequence at the amino terminal end of the fusion protein, that directs at least the portion of the fusion protein including the test polypeptide to be secreted from the cytosol of a cell and localized on the extracellular side of the cell membrane.

Gene constructs encoding fusion proteins are likewise referred to a “chimeric genes” or “fusion genes”.

The term “vector” refers to a DNA molecule, capable of replication in a host cell, into which a gene can be inserted to construct a recombinant DNA molecule.

The terms “phage vector” and “phagemid” are art-recognized and generally refer to a vector derived by modification of a phage genome, containing an origin of replication for a bacteriophage, and preferably, though optional, an origin (ori) for a bacterial plasmid. The use of phage vectors rather than the phage genome itself provides greater flexibility to vary the ratio of chimeric polypeptide/coat protein to wild-type coat protein, as well as supplement the phage genes with additional genes encoding other heterologous polypeptides, such as “auxiliary polypeptides” which may be useful in the “dual” polypeptide display constructs described below.

The language “helper phage” describes a phage which is used to infect cells containing a defective phage genome or phage vector and which functions to complement the defect. The defect can be one which results from removal or inactivation of phage genomic sequence required for production of phage particles. Examples of helper phage are M13K07.

As used herein, a “reporter gene construct” is a nucleic acid that includes a “reporter gene” operatively linked to at least one transcriptional regulatory sequence. Transcription of the reporter gene is controlled by these sequences to which they are linked.

The term “sequester”, as used herein, means to separate, segregate, remove, or bind a lipid kinase inhibitor complex, e.g., on a solid support. In preferred embodiments, a lipid kinase inhibitor complex is sequestered by a solid support such that other non-sequestered LBPs can be removed, e.g., by washing or other purification techniques. A lipid kinase inhibitor complex is “reversibly sequestered” if the process of sequestering the complex on a solid support can be reversed to yield a free complex or free LBP, e.g., in solution in a reaction mixture. In preferred embodiments, the process of sequestering a complex, or of reversing the sequestration, or both, occurs under mild conditions and in high yield, e.g., greater than at least about 40% yield.

The term “polymeric support”, as used herein, refers to a soluble or insoluble polymer to which a lipid kinase inhibitor can be covalently bonded (e.g., by through an ester functionality) by reaction with a functional group of the polymeric support. Many suitable polymeric supports are known, and include soluble polymers such as polyethylene glycols or polyvinyl alcohols, as well as insoluble polymers such as polystyrene resins. A suitable polymeric support includes functional groups such as those described below. A polymeric support is termed “soluble” if a polymer, or a polymer-supported compound, is soluble under the conditions employed. However, in general, a soluble polymer can be rendered insoluble under defined conditions. Accordingly, a polymeric support can be soluble under certain conditions and insoluble under other conditions. A polymeric support is termed “insoluble” if reaction of a lipid kinase inhibitor with the polymeric support results in an insoluble polymer-supported lipid kinase inhibitor under the conditions employed.

The term “therapeutic profiling”, as used herein, refers to the profiling of compounds for clinical suitability. The term therapeutic profiling includes, but is not limited to, efficacy testing and toxicity screening.

The term “wortmannin analog”, as used herein, refers to any derivative of wortmannin. Examples of wortmannin analogs include, but are not limited to, biotin-wortmannin (See Example 1) and BODIPY-wortmannin (See Example 4).

The term “wortmannin bait moiety” or “wortmannin moiety” as used herein includes moieties of wortmannin or a wortmannin analog.

Abbreviations used herein include: ARF—ADP ribosylation factor; Btk—Brutons tyrosine kinase; DTT—dithiothreitol; Erk/MAPK—extracellular regulated kinase/mitogen activated protein kinase; EST—expressed sequence tag; GAP=GTPase activating protein; GAPDH=glyceraldehyde 3-phosphate dehydrogenase; GTP—guanosine triphosphate; HEPES—(N-[2-hydroxyethyl]piperazine-N′-[2-ethanesulfonic acid); kb=kilobase; SDS—sodium dodecyl sulfate; MBP=maltose binding protein; MBP-PH=maltose binding protein Akt PH domain fusion protein; NP-40—nonylphenylpolyethylene glycol; 3′PPI—3′ phosphorylated phosphatidylinositols; PAGE—polyacrylamide gel electrophoresis; PCR—polymerase chain reaction; PDK1—phosphoinositide dependent kinase 1; PH=pleckstrin homology; PI3′-K=phosphatidylinositol 3′-kinase; PKC—protein kinase C; PLA—phopholipase A; PLCγ—phospholipase Cγ; PtdIns—phosphatidylinositol; PtdIns-3-P—phosphatidylinositol-3-monophosphate; PtdIns-3,4-P₂—phosphatidylinositol-3,4-bisphosphate; PtdIns-4,5-P₂—phosphatidylinositol-4,5-bisphosphate; PtdIns-3,4,5-P₃—phosphatidylinositol-3,4,5-trisphosphate; SDS—sodium dodecyl sulfate; SH2=Src homology 2.

C) Exemplary Embodiments of Lipid Kinase Inhibitor Baits

As set forth above, in certain embodiments, the subject method can be practiced by utilizing immobilized lipid kinase inhibitor moieties, such as wortmannin or analogs thereof, as the bait for identifying polypeptides or other cellular components capable of interacting with, and forming complexes with the lipid kinase inhibitor moiety. In certain embodiments, the subject lipid kinase inhibitor moiety is a covalent inhibitor of a phosphatidylinositol kinase. Exemplary lipid kinase inhibitor moieties include:

Wortmannin,

Hydroxywortmannin,

LY294002,

Demethoxyviridin,

Quercetin,

myricetin, and

staurosporine.

In certain preferred embodiments, the subject lipid kinase inhibitor can be immobilized or incorporated into a polymer or other insoluble matrix by, for example, derivativation with one or more of subject lipid kinase inhibitor moieties derivatized to a solid support, such as glass, silicon, or a polymeric support. The support can be, inter alia, a bead, a chip, a hydrogel, etc.

In certain preferred embodiments, the subject lipid kinase inhibitor moieties are derivatized by covalent or non-covalent coupling. For example, the present invention specifically contemplates bait moieties represented in the general formula

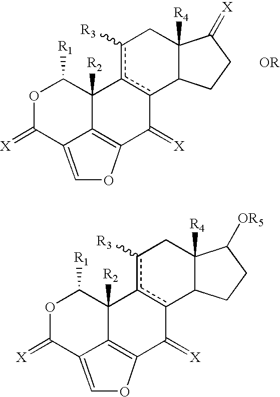

wherein

X, independently for each occurrence, represents O or S,

R₁ represents —(CH₂)ₙ—X—R₅,

R₂, R₄ and R₅, independently, represent H or a C1-C6 alkyl,

R₃ represents S-L-,

S represents a solid support or molecular or chemical tag for purifying or identifying the bait moiety,

L represents a linker, and

n is 0, 1, 2 or 3.

In certain preferred embodiments, X represents O; and L is a linker of 150-1500 amu, such as PEG.

In other preferred embodiments, the bait moiety is represented by the formula:

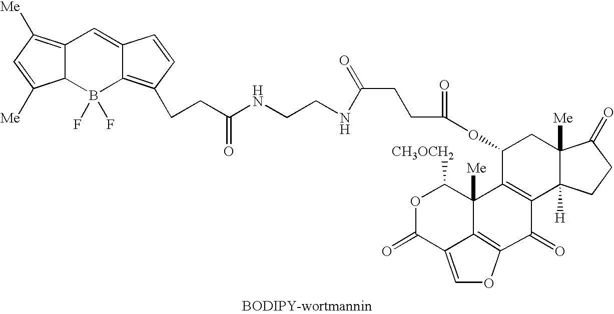

In certain embodiments, particularly where more than one type of lipid kinase inhibitor moiety is used as a bait (e.g., a library of different lipid kinase inhibitor moieties), a spatial array of lipid kinase inhibitor baits can be generated, e.g., for library versus library screening. For example, libraries of at least 10 different lipid kinase inhibitor moieties can be tested as baits, and more preferably libraries of at least 100 or even 1000 different lipid kinase inhibitor moieties.

The lipid kinase inhibitor moiety can be derivatived to the support by any of a number of means. As described in the appended examples, biotinylation of the phosphate head group can be used to derivatize the lipid kinase inhibitor moiety to an avidin-displaying support. In this case, there is a need to link the lipid kinase inhibitor to the solid support or chemical or molecular tag.

There are a large number of other chemical cross-linking agents known in the art which could be used in the present invention for this purpose. For the present invention, the preferred cross-linking agents are heterobifunctional cross-linkers, which can be used to link the lipid kinase inhibitor bait and solid support in a stepwise manner. Heterobifunctional cross-linkers provide the ability to design more specific coupling methods for conjugating the subject moieties, thereby reducing the occurrences of unwanted side reactions such as homo-lipid kinase inhibitor polymers. A wide variety of heterobifunctional cross-linkers are known in the art. These include: succinimidyl 4-(N-maleimidomethyl) cyclohexane-1-carboxylate (SMCC), m-Maleimidobenzoyl-N-hydroxysuccinimide ester (MBS); N-succinimidyl (4-iodoacetyl) aminobenzoate (SIAB), succinimidyl 4-(p-maleimidophenyl) butyrate (SMPB), 1-ethyl-3-(3-dimethylaminopropyl)carbodiimide hydrochloride (EDC); 4-succinimidyloxycarbonyl-a-methyl-a-(2-pyridyldithio)-tolune (SMPT), N-succinimidyl 3-(2-pyridyldithio)propionate (SPDP), succinimidyl 6-3-(2-pyridyldithio)propionate hexanoate (LC-SPDP). Those cross-linking agents having N-hydroxysuccinimide moieties can be obtained as the N-hydroxysulfosuccinimide analogs, which generally have greater water solubility. In addition, those cross-linking agents having disulfide bridges within the linking chain can be synthesized instead as the alkyl derivatives so as to reduce the amount of linker cleavage in vivo.

In addition to the heterobifunctional cross-linkers, there exists a number of other useful cross-linking agents including homobifunctional and photoreactive cross-linkers. Disuccinimidyl suberate (DSS), bismaleimidohexane (BMH) and dimethylpimelimidate-2 HCl (DMP) are examples of useful homobifunctional cross-linking agents, and bis-β-(4-azidosalicylamido) ethyldisulfide (BASED) and N-succinimidyl-6 (4′-azido-2′-nitrophenylamino) hexanoate (SANPAH) are examples of useful photoreactive cross-linkers for use in this invention. For a review of coupling techniques which may be applied to the subject lipid kinase inhibitor moieties, see Means et al. (1990) Bioconjugate Chemistry 1:2-12.

The third component of the heterobifunctional cross-linker is the spacer arm or bridge. The bridge is the structure that connects the two reactive ends. The most apparent attribute of the bridge is its effect on steric hindrance. In some instances, a longer bridge can more easily span the distance necessary to link two complex biomolecules. For instance, SMPB has a span of 14.5 angstroms.

D) Exemplary Embodiments of Polypeptide Libraries

One goal of the present method is to identify proteins which are bound by the lipid kinase inhibitor bait moiety. Accordingly, the present invention contemplates that any of a number of methods for trapping, sequestering, or identifying protein complexes using a lipid kinase inhibitor bait moiety. For instance, the proteins bound to the lipid kinase inhibitor bait can be identified by sequencing using mass spectroscopy or Edman sequencing. This technique can be advantageous when the source of test proteins is a cell lysate. In other embodiments, proteins bound to the lipid kinase inhibitor bait can be identified using gel electrophoresis, preferentially two-dimensional gel electrophoresis. In other embodiments, the polypeptides are associated with a tag(s) which identifies the sequence of the protein, or with the gene which encodes the protein. In still other instance, the proteins are provided as part of a spatial array for which the coordinates on the array provides the identity of the protein.

In certain preferred embodiments, the polypeptide library is provided as an expression library. For instance, a library of test polypeptides is expressed by a population of display packages to form a peptide display library. With respect to the display package on which the variegated peptide library is manifest, it will be appreciated from the discussion provided herein that the display package will preferably be able to be (i) genetically altered to encode heterologous peptide, (ii) maintained and amplified in culture, (iii) manipulated to display the peptide-containing gene product in a manner permitting the peptide to interact with a lipid kinase inhibitor during an affinity separation step, and (iv) affinity separated while retaining the nucleotide sequence encoding the test polypeptide (herein “peptide gene”) such that the sequence of the peptide gene can be obtained. In preferred embodiments, the display remains viable after affinity separation.

Ideally, the display package comprises a system that allows the sampling of very large variegated peptide display libraries, rapid sorting after each affinity separation round, and easy isolation of the peptide gene from purified display packages or further manipulation of that sequence in the secretion mode. The most attractive candidates for this type of screening are prokaryotic organisms and viruses, as they can be amplified quickly, they are relatively easy to manipulate, and large number of clones can be created. Preferred display packages include, for example, vegetative bacterial cells, bacterial spores, and most preferably, bacterial viruses (especially DNA viruses). However, the present invention also contemplates the use of eukaryotic cells, including yeast and their spores, as potential display packages.

In addition to commercially available kits for generating phage display libraries (e.g. the Pharmacia Recombinant Phage Antibody System, catalog no. 27-9400-01; and the Stratagene SurFZAP™ phage display kit, catalog no. 240612), examples of methods and reagents particularly amenable for use in generating the variegated peptide display library of the present invention can be found in, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; the Ladner et al. International Publication No. WO 90/02809; Fuchs et al. (1991) Bio/Technology 9:1370-1372; Hay et al. (1992) Hum Antibod Hybridomas 3:81-85; Huse et al. (1989) Science 246:1275-1281; Griffths et al. (1993) EMBO J. 12:725-734; Hawkins et al. (1992) J Mol Biol 226:889-896; Clackson et al. (1991) Nature 352:624-628; Gram et al. (1992) PNAS 89:3576-3580; Garrad et al. (1991) Bio/Technology 9:1373-1377; Hoogenboom et al. (1991) Nuc Acid Res 19:4133-4137; and Barbas et al. (1991) PNAS 88:7978-7982. These systems can, with modifications described herein, be adapted for use in the subject method.

When the display is based on a bacterial cell, or a phage which is assembled periplasmically, the display means of the package will comprise at least two components. The first component is a secretion signal which directs the recombinant peptide to be localized on the extracellular side of the cell membrane (of the host cell when the display package is a phage). This secretion signal can be selected so as to be cleaved off by a signal peptidase to yield a processed, “mature” peptide. The second component is a display anchor protein which directs the display package to associate the test polypeptide with its outer surface. As described below, this anchor protein can be derived from a surface or coat protein native to the genetic package.

When the display package is a bacterial spore, or a phage whose protein coating is assembled intracellularly, a secretion signal directing the peptide to the inner membrane of the host cell is unnecessary. In these cases, the means for arraying the variegated peptide library comprises a derivative of a spore or phage coat protein amenable for use as a fusion protein.

In some instances it may be necessary to introduce an unstructured polypeptide linker region between portions of the chimeric protein, e.g., between the test polypeptide and display polypeptide. This linker can facilitate enhanced flexibility of the chimeric protein allowing the test polypeptide to freely interact with a lipid kinase inhibitor by reducing steric hindrance between the two fragments, as well as allowing appropriate folding of each portion to occur. The linker can be of natural origin, such as a sequence determined to exist in random coil between two domains of a protein. Alternatively, the linker can be of synthetic origin. For instance, the sequence (Gly₄Ser)₃ can be used as a synthetic unstructured linker. Linkers of this type are described in Huston et al. (1988) PNAS 85:4879; and U.S. Pat. Nos. 5,091,513 and 5,258,498. Naturally occurring unstructured linkers of human origin are preferred as they reduce the risk of immunogenicity.

In the instance wherein the display package is a phage, the cloning site for the test polypeptide gene sequences in the phagemid should be placed so that it does not substantially interfere with normal phage function. One such locus is the intergenic region as described by Zinder and Boeke, (1982) Gene 19:1-10.

The number of possible combinations in a peptide library can get large as the length is increased and selection criteria for degenerating at each position is relaxed. To sample as many combinations as possible depends, in part, on the ability to recover large numbers of transformants. For phage with plasmid-like forms (as filamentous phage), electrotransformation provides an efficiency comparable to that of phage-transfection with in vitro packaging, in addition to a very high capacity for DNA input. This allows large amounts of vector DNA to be used to obtain very large numbers of transformants. The method described by Dower et al. (1988) Nucleic Acids Res., 16:6127-6145, for example, may be used to transform fd-tet derived recombinants at the rate of about 10⁷ transformants/ug of ligated vector into E. coli (such as strain MC1061), and libraries may be constructed in fd-tet B1 of up to about 3×10⁸ members or more. Increasing DNA input and making modifications to the cloning protocol within the ability of the skilled artisan may produce increases of greater than about 10-fold in the recovery of transformants, providing libraries of up to 10¹⁰ or more recombinants.

As will be apparent to those skilled in the art, in embodiments wherein high affinity peptides are sought, an important criteria for the present selection method can be that it is able to discriminate between peptides of different affinity for a particular lipid kinase inhibitor, and preferentially enrich for the peptides of highest affinity. Applying the well known principles of peptide affinity and valence (i.e. avidity), it is understood that manipulating the display package to be rendered effectively monovalent can allow affinity enrichment to be carried out for generally higher binding affinities (i.e. binding constants in the range of 10⁶ to 10¹⁰ M⁻¹) as compared to the broader range of affinities isolable using a multivalent display package. To generate the monovalent display, the natural (i.e. wild-type) form of the surface or coat protein used to anchor the peptide to the display can be added at a high enough level that it almost entirely eliminates inclusion of the peptide fusion protein in the display package. Thus, a vast majority of the display packages can be generated to include no more than one copy of the peptide fusion protein (see, for example, Garrad et al. (1991) Bio/Technology 9:1373-1377). In a preferred embodiment of a monovalent display library, the library of display packages will comprise no more than 5 to 10% polyvalent displays, and more preferably no more than 2% of the display will be polyvalent, and most preferably, no more than 1% polyvalent display packages in the population. The source of the wild-type anchor protein can be, for example, provided by a copy of the wild-type gene present on the same construct as the peptide fusion protein, or provided by a separate construct altogether. However, it will be equally clear that by similar manipulation, polyvalent displays can be generated to isolate a broader range of binding affinities. Such peptides can be useful, for example, in purification protocols where avidity can be desirable.

i) Phages As Display Packages

Bacteriophage are attractive prokaryotic-related organisms for use in the subject method. Bacteriophage are excellent candidates for providing a display system of the variegated polypeptide library as there is little or no enzymatic activity associated with intact mature phage, and because their genes are inactive outside a bacterial host, rendering the mature phage particles metabolically inert. In general, the phage surface is a relatively simple structure. Phage can be grown easily in large numbers, they are amenable to the practical handling involved in many potential mass screening programs, and they carry genetic information for their own synthesis within a small, simple package. As the polypeptide gene is inserted into the phage genome, choosing the appropriate phage to be employed in the subject method will generally depend most on whether (i) the genome of the phage allows introduction of the polypeptide gene either by tolerating additional genetic material or by having replaceable genetic material; (ii) the virion is capable of packaging the genome after accepting the insertion or substitution of genetic material; and (iii) the display of the polypeptide on the phage surface does not disrupt virion structure sufficiently to interfere with phage propagation.

One concern presented with the use of phage is that the morphogenetic pathway of the phage determines the environment in which the polypeptide will have opportunity to fold. Periplasmically assembled phage are preferred as the displayed polypeptides may contain essential disulfides, and such polypeptides may not fold correctly within a cell. However, in certain embodiments in which the display package forms intracellularly (e.g., where λ phage are used), it has been demonstrated in other instances that disulfide-containing polypeptides can assume proper folding after the phage is released from the cell.

Another concern related to the use of phage, but also pertinent to the use of bacterial cells and spores as well, is that multiple infections could generate hybrid displays that carry the gene for one particular test polypeptide yet have two or more different test polypeptides on their surfaces. Therefore, it can be preferable, though optional, to minimize this possibility by infecting cells with phage under conditions resulting in a low multiple-infection.

For a given bacteriophage, the preferred display means is a protein that is present on the phage surface (e.g. a coat protein). Filamentous phage can be described by a helical lattice; isometric phage, by an icosahedral lattice. Each monomer of each major coat protein sits on a lattice point and makes defined interactions with each of its neighbors. Proteins that fit into the lattice by making some, but not all, of the normal lattice contacts are likely to destabilize the virion by aborting formation of the virion as well as by leaving gaps in the virion so that the nucleic acid is not protected. Thus in bacteriophage, unlike the cases of bacteria and spores, it is generally important to retain in the polypeptide fusion proteins those residues of the coat protein that interact with other proteins in the virion. For example, when using the M13 cpVIII protein, the entire mature protein will generally be retained with the polypeptide fragment being added to the N-terminus of cpVIII, while on the other hand it can suffice to retain only the last 100 carboxy terminal residues (or even fewer) of the M13 cpIII coat protein in the polypeptide fusion protein.

Under the appropriate induction, the test polypeptide library is expressed and exported, as part of the fusion protein, to the bacterial cytoplasm, such as when the λ phage is employed. The induction of the fusion protein(s) may be delayed until some replication of the phage genome, synthesis of some of the phage structural-proteins, and assembly of some phage particles has occurred. The assembled protein chains then interact with the phage particles via the binding of the anchor protein on the outer surface of the phage particle. The cells are lysed and the phage bearing the library-encoded test polypeptides (that corresponds to the specific library sequences carried in the DNA of that phage) are released and isolated from the bacterial debris.

To enrich for and isolate phage which encodes a selected test polypeptide, and thus to ultimately isolate the nucleic acid sequences (the polypeptide gene) themselves, phage harvested from the bacterial debris are affinity purified. As described below, when a test polypeptide which specifically binds a particular lipid kinase inhibitor is desired, the lipid kinase inhibitor can be used to retrieve phage displaying the desired test polypeptide. The phage so obtained may then be amplified by infecting into host cells. Additional rounds of affinity enrichment followed by amplification may be employed until the desired level of enrichment is reached.

The enriched polypeptide-phage can also be screened with additional detection-techniques such as expression plaque (or colony) lift (see, e.g., Young and Davis, Science (1983) 222:778-782) whereby a labeled lipid kinase inhibitor is used as a probe.

a) Filamentous Phage

Filamentous bacteriophages, which include M13, fl, fd, Ifl, Ike, Xf, Pfl, and Pf3, are a group of related viruses that infect bacteria. They are termed filamentous because they are long, thin particles comprised of an elongated capsule that envelopes the deoxyribonucleic acid (DNA) that forms the bacteriophage genome. The F pili filamentous bacteriophage (Ff phage) infect only gram-negative bacteria by specifically adsorbing to the tip of F pili, and include fd, fl and M13.

Compared to other bacteriophage, filamentous phage in general are attractive and M13 in particular is especially attractive because: (i) the 3-D structure of the virion is known; (ii) the processing of the coat protein is well understood; (iii) the genome is expandable; (iv) the genome is small; (v) the sequence of the genome is known; (vi) the virion is physically resistant to shear, heat, cold, urea, guanidinium chloride, low pH, and high salt; (vii) the phage is a sequencing vector so that sequencing is especially easy; (viii) antibiotic-resistance genes have been cloned into the genome with predictable results (Hines et al. (1980) Gene 11:207-218); (ix) it is easily cultured and stored, with no unusual or expensive media requirements for the infected cells, (x) it has a high burst size, each infected cell yielding 100 to 1000 M13 progeny after infection; and (xi) it is easily harvested and concentrated (Salivar et al. (1964) Virology 24: 359-371). The entire life cycle of the filamentous phage M13, a common cloning and sequencing vector, is well understood. The genetic structure of M13 is well known, including the complete sequence (Schaller et al. in The Single-Stranded DNA Phages eds. Denhardt et al. (NY: CSHL Press, 1978)), the identity and function of the ten genes, and the order of transcription and location of the promoters, as well as the physical structure of the virion (Smith et al. (1985) Science 228:1315-1317; Raschad et al. (1986) Microbiol Dev 50:401-427; Kuhn et al. (1987) Science 238:1413-1415; Zimmerman et al. (1982) J Biol Chem 257:6529-6536; and Banner et al. (1981) Nature 289:814-816). Because the genome is small (6423 bp), cassette mutagenesis is practical on RF M13 (Current Protocols in Molecular Biology, eds. Ausubel et al. (NY: John Wiley & Sons, 1991)), as is single-stranded oligonucleotide directed mutagenesis (Fritz et al. in DNA Cloning, ed by Glover (Oxford, UK: IRC Press, 1985)). M13 is a plasmid and transformation system in itself, and an ideal sequencing vector. M13 can be grown on Rec-strains of E. coli. The Ml 3 genome is expandable (Messing et al. in The Single-Stranded DNA Phages, eds Denhardt et al. (NY: CSHL Press, 1978) pages 449-453; and Fritz et al., supra) and M13 does not lyse cells. Extra genes can be inserted into M13 and will be maintained in the viral genome in a stable manner.

The mature capsule or Ff phage is comprised of a coat of five phage-encoded gene products: cpVIII, the major coat protein product of gene VIII that forms the bulk of the capsule; and four minor coat proteins, cpIII and cpIV at one end of the capsule and cpVII and cpIX at the other end of the capsule. The length of the capsule is formed by 2500 to 3000 copies of cpVIII in an ordered helix array that forms the characteristic filament structure. The gene III-encoded protein (cpIII) is typically present in 4 to 6 copies at one end of the capsule and serves as the receptor for binding of the phage to its bacterial host in the initial phase of infection. For detailed reviews of Ff phage structure, see Rasched et al., Microbiol. Rev., 50:401-427 (1986); and Model et al., in The Bacteriophages, Volume 2, R. Calendar, Ed., Plenum Press, pp. 375-456 (1988).

The phage particle assembly involves extrusion of the viral genome through the host cell's membrane. Prior to extrusion, the major coat protein cpVIII and the minor coat protein cpIII are synthesized and transported to the host cell's membrane. Both cpVIII and cpIII are anchored in the host cell membrane prior to their incorporation into the mature particle. In addition, the viral genome is produced and coated with cpV protein. During the extrusion process, cpV-coated genomic DNA is stripped of the cpV coat and simultaneously recoated with the mature coat proteins.

Both cpIII and cpVIII proteins include two domains that provide signals for assembly of the mature phage particle. The first domain is a secretion signal that directs the newly synthesized protein to the host cell membrane. The secretion signal is located at the amino terminus of the polypeptide and lipid kinase inhibitors the polypeptide at least to the cell membrane. The second domain is a membrane anchor domain that provides signals for association with the host cell membrane and for association with the phage particle during assembly. This second signal for both cpVIII and cpIII comprises at least a hydrophobic region for spanning the membrane.

The 50 amino acid mature gene VIII coat protein (cpVIII) is synthesized as a 73 amino acid precoat (Ito et al. (1979) PNAS 76:1199-1203). cpVIII has been extensively studied as a model membrane protein because it can integrate into lipid kinase inhibitor bilayers such as the cell membrane in an asymmetric orientation with the acidic amino terminus toward the outside and the basic carboxy terminus toward the inside of the membrane. The first 23 amino acids constitute a typical signal-sequence which causes the nascent polypeptide to be inserted into the inner cell membrane. An E. Coli signal peptidase (SP-I) recognizes amino acids 18, 21, and 23, and, to a lesser extent, residue 22, and cuts between residues 23 and 24 of the precoat (Kuhn et al. (1985) J. Biol. Chem. 260:15914-15918; and Kuhn et al. (1985) J. Biol. Chem. 260:15907-15913). After removal of the signal sequence, the amino terminus of the mature coat is located on the periplasmic side of the inner membrane; the carboxy terminus is on the cytoplasmic side. About 3000 copies of the mature coat protein associate side-by-side in the inner membrane.

The sequence of gene VIII is known, and the amino acid sequence can be encoded on a synthetic gene. Mature gene VIII protein makes up the sheath around the circular ssDNA. The gene VIII protein can be a suitable anchor protein because its location and orientation in the virion are known (Banner et al. (1981) Nature 289:814-816). Preferably, the polypeptide is attached to the amino terminus of the mature M13 coat protein to generate the phage display library. As set out above, manipulation of the concentration of both the wild-type cpVIII and Ab/cpVIII fusion in an infected cell can be utilized to decrease the avidity of the display and thereby enhance the detection of high affinity polypeptides directed to the lipid kinase inhibitor(s).

Another vehicle for displaying the polypeptide is by expressing it as a domain of a chimeric gene containing part or all of gene III, e.g., encoding cpIII. When monovalent displays are required, expressing the polypeptide as a fusion protein with cpIII can be a preferred embodiment, as manipulation of the ratio of wild-type cpIII to chimeric cpIII during formation of the phage particles can be readily controlled. This gene encodes one of the minor coat proteins of M13. Genes VI, VII, and IX also encode minor coat proteins. Each of these minor proteins is present in about 5 copies per virion and is related to morphogenesis or infection. In contrast, the major coat protein is present in more than 2500 copies per virion. The gene VI, VII, and IX proteins are present at the ends of the virion; these three proteins are not posttranslationally processed (Rasched et al. (1986) Ann Rev. Microbiol. 41:507-541). In particular, the single-stranded circular phage DNA associates with about five copies of the gene III protein and is then extruded through the patch of membrane-associated coat protein in such a way that the DNA is encased in a helical sheath of protein (Webster et al. in The Single-Stranded DNA Phages, eds Dressler et al. (NY:CSHL Press, 1978).

Manipulation of the sequence of cpII has demonstrated that the C-terminal 23 amino acid residue stretch of hydrophobic amino acids normally responsible for a membrane anchor function can be altered in a variety of ways and retain the capacity to associate with membranes. Ff phage-based expression vectors were first described in which the cpIII amino acid residue sequence was modified by insertion of heterologous polypeptide (Parmely et al., Gene (1988) 73:305-318; and Cwirla et al., PNAS (1990) 87:6378-6382) or an amino acid residue sequence defining a single chain polypeptide domain (McCafferty et al., Science (1990) 348:552-554). It has been demonstrated that insertions into gene III can result in the production of novel protein domains on the virion outer surface. (Smith (1985) Science 228:1315-1317; and de la Cruz et al. (1988) J. Biol. Chem. 263:4318-4322). The polypeptide gene may be fused to gene III at the site used by Smith and by de la Cruz et al., at a codon corresponding to another domain boundary or to a surface loop of the protein, or to the amino terminus of the mature protein.

Generally, the successful cloning strategy utilizing a phage coat protein, such as cpIII of filamentous phage fd, will provide expression of a polypeptide chain fused to the N-terminus of a coat protein (e.g., cpIII) and transport to the inner membrane of the host where the hydrophobic domain in the C-terminal region of the coat protein anchors the fusion protein in the membrane, with the N-terminus containing the polypeptide chain protruding into the periplasmic space.

Similar constructions could be made with other filamentous phage. Pf3 is a well known filamentous phage that infects Pseudomonos aerugenosa cells that harbor an IncP-I plasmid. The entire genome has been sequenced ((Luiten et al. (1985) J. Virol. 56:268-276) and the genetic signals involved in replication and assembly are known (Luiten et al. (1987) DNA 6:129-137). The major coat protein of PF3 is unusual in having no signal peptide to direct its secretion. The sequence has charged residues ASP-7, ARG-37, LYS-40, and PHE44 which is consistent with the amino terminus being exposed. Thus, to cause a polypeptide to appear on the surface of Pf3, a tripartite gene can be constructed which comprises a signal sequence known to cause secretion in P. aerugenosa, fused in-frame to a gene fragment encoding the polypeptide sequence, which is fused in-frame to DNA encoding the mature Pf3 coat protein. Optionally, DNA encoding a flexible linker of one to 10 amino acids is introduced between the polypeptide gene fragment and the Pf3 coat-protein gene. This tripartite gene is introduced into Pf3 so that it does not interfere with expression of any Pf3 genes. Once the signal sequence is cleaved off, the polypeptide is in the periplasm and the mature coat protein acts as an anchor and phage-assembly signal.

b) Bacteriophage φX174

The bacteriophage φX174 is a very small icosahedral virus which has been thoroughly studied by genetics, biochemistry, and electron microscopy (see The Single Stranded DNA Phages (eds. Den hardt et al. (NY:CSHL Press, 1978)). Three gene products of φ174 are present on the outside of the mature virion: F (capsid), G (major spike protein, 60 copies per virion), and H (minor spike protein, 12 copies per virion). The G protein comprises 175 amino acids, while H comprises 328 amino acids. The F protein interacts with the single-stranded DNA of the virus. The proteins F, G, and H are translated from a single mRNA in the viral infected cells. As the virus is so tightly constrained because several of its genes overlap, φX174 is not typically used as a cloning vector due to the fact that it can accept very little additional DNA. However, mutations in the viral G gene (encoding the G protein) can be rescued by a copy of the wild-type G gene carried on a plasmid that is expressed in the same host cell (Chambers et al. (1982) Nuc Acid Res 10:6465-6473). In one embodiment, one or more stop codons are introduced into the G gene so that no G protein is produced from the viral genome. The variegated polypeptide gene library can then be fused with the nucleic acid sequence of the H gene. An amount of the viral G gene equal to the size of polypeptide gene fragment is eliminated from the φX174 genome, such that the size of the genome is ultimately unchanged. Thus, in host cells also transformed with a second plasmid expressing the wild-type G protein, the production of viral particles from the mutant virus is rescued by the exogenous G protein source. Where it is desirable that only one test polypeptide be displayed per φX174 particle, the second plasmid can further include one or more copies of the wild-type H protein gene so that a mix of H and test polypeptide/H proteins will be predominated by the wild-type H upon incorporation into phage particles.

c) Large DNA Phage

Phage such as λ or T4 have much larger genomes than do M13 or φX174, and have more complicated 3-D capsid structures than M13 or φPX174, with more coat proteins to choose from. In embodiments of the invention whereby the test polypeptide library is processed and assembled into a functional form and associates with the bacteriophage particles within the cytoplasm of the host cell, bacteriophage λ and derivatives thereof are examples of suitable vectors. The intracellular morphogenesis of phage λ can potentially prevent protein domains that ordinarily contain disulfide bonds from folding correctly. However, variegated libraries expressing a population of functional polypeptides, which include such bonds, have been generated in λ phage. (Huse et al. (1989) Science 246:1275-1281; Mullinax et al. (1990) PNAS 87:8095-8099; and Pearson et al. (1991) PNAS 88:2432-2436). Such strategies take advantage of the rapid construction and efficient transformation abilities of λ phage.

When used for expression of polypeptide sequences (ixogenous nucleotide sequences), may be readily inserted into a λ vector. For instance, variegated polypeptide libraries can be constructed by modification of λ ZAP II through use of the multiple cloning site of a λ ZAP II vector (Huse et al. supra).

ii) Bacterial Cells as Display Packages

Recombinant polypeptides are able to cross bacterial membranes after the addition of appropriate secretion signal sequences to the N-terminus of the protein (Better et al (1988) Science 240:1041-1043; and Skerra et al. (1988) Science 240:1038-1041). In addition, recombinant polypeptides have been fused to outer membrane proteins for surface presentation. For example, one strategy for displaying polypeptides on bacterial cells comprises generating a fusion protein by inserting the polypeptide into cell surface exposed portions of an integral outer membrane protein (Fuchs et al. (1991) Bio/Technology 9:1370-1372). In selecting a bacterial cell to serve as the display package, any well-characterized bacterial strain will typically be suitable, provided the bacteria may be grown in culture, engineered to display the test polypeptide library on its surface, and is compatible with the particular affinity selection process practiced in the subject method. Among bacterial cells, the preferred display systems include Salmonella typhirnurium, Bacillus subtilis, Pseudomonas aeruginosa, Vibrio cholerae, Klebsiella pneumonia, Neisseria gonorrhoeae, Neisseria meningitidis, Bacteroides nodosus, Moraxella bovis, and especially Escherichia coli. Many bacterial cell surface proteins useful in the present invention have been characterized, and works on the localization of these proteins and the methods of determining their structure include Benz et al. (1988) Ann Rev Microbiol 42: 359-393; Balduyck et al. (1985) Biol Chem Hoppe-Seyler 366:9-14; Ehrmann et al (1990) PNAS 87:7574-7578; Heijne et al. (1990) Protein Engineering 4:109-112; Ladner et al. U.S. Pat. No. 5,223,409; Ladner et al. WO88/06630; Fuchs et al. (1991) Bio/technology 9:1370-1372; and Goward et al. (1992) TIBS 18:136-140.

To further illustrate, the LamB protein of E coli is a well understood surface protein that can be used to generate a variegated library of test polypeptides on the surface of a bacterial cell (see, for example, Ronco et al. (1990) Biochemie 72:183-189; van der Weit et al. (1990) Vaccine 8:269-277; Charabit et al. (1988) Gene 70:181-189; and Ladner U.S. Pat. No. 5,222,409). LamB of E. coli is a porin for maltose and maltodextrin transport, and serves as the receptor for adsorption of bacteriophages X and K10. LamB is transported to the outer membrane if a functional N-terminal signal sequence is present (Benson et al. (1984) PNAS 81:3830-3834). As with other cell surface proteins, LamB is synthesized with a typical signal-sequence which is subsequently removed. Thus, the variegated polypeptide gene library can be cloned into the LamB gene such that the resulting library of fusion proteins comprise a portion of LamB sufficient to anchor the protein to the cell membrane with the test polypeptide fragment oriented on the extracellular side of the membrane. Secretion of the extracellular portion of the fusion protein can be facilitated by inclusion of the LamB signal sequence, or other suitable signal sequence, as the N-terminus of the protein.

The E. coli LamB has also been expressed in functional form in S. typhimurium (Harkki et al. (1987) Mol Gen Genet 209:607-611), V. cholerae (Harkki et al. (1986) Microb Pathol 1:283-288), and K. pneumonia (Wehmeier et al. (1989) Mol Gen Genet 215:529-536), so that one could display a population of test polypeptides in any of these species as a fusion to E. coli LamB. Moreover, K. pneumonia expresses a maltoporin similar to LamB which could also be used. In P. aeruginosa, the D1 protein (a homologue of LamB) can be used (Trias et al. (1988) Biochem Biophys Acta 938:493-496). Similarly, other bacterial surface proteins, such as PAL, OmpA, OmpC, OmpF, PhoE, pilin, BtuB, FepA, FhuA, IutA, FecA and FhuE, may be used in place of LamB as a portion of the display means in a bacterial cell.

In another exemplary embodiment, the fusion protein can be derived using the FliTrx™ Random Polypeptide Display Library (Invitrogen). That library is a diverse population of random dodecapolypeptides inserted within the thioredoxin active-site loop inside the dispensable region of the bacterial flagellin gene (fliC). The resultant recombinant fusion protein (FLITRX) is exported and assembled into partially functional flagella on the bacterial cell surface, displaying the random polypeptide library.

Polypeptides are fused in the middle of thioredoxin, therefore, both their N- and C-termini are anchored by thioredoxin's tertiary structure. This results in the display of a constrained polypeptide. By contrast, phage display proteins are fused to the N-terminus of phage coat proteins in an unconstrained manner. The unconstrained molecules possess many degrees of conformational freedom which may result in the lack of proper interaction with the lipid kinase inhibitor molecule. Without proper interaction, many potential protein-protein interactions may be missed.

Moreover, phage display is limited by the low expression levels of bacteriophage coat proteins. FliTrx™ and similar methods can overcome this limitation by using a strong promoter to drive expression of the test polypeptide fusions that are displayed as multiple copies.

According to the present invention, it is contemplated that the FliTrx vector can be modified to provide a vector which is differentially spliced in mammalian cells to yield a secreted, soluble test polypeptide.

iii) Bacterial Spores as Display Packages

Bacterial spores also have desirable properties as display package candidates in the subject method. For example, spores are much more resistant than vegetative bacterial cells or phage to chemical and physical agents, and hence permit the use of a great variety of affinity selection conditions. Also, Bacillus spores neither actively metabolize nor alter the proteins on their surface. However, spores have the disadvantage that the molecular mechanisms that trigger sporulation are less well worked out than is the formation of M13 or the export of protein to the outer membrane of E. coli, though such a limitation is not a serious detractant from their use in the present invention.

Bacteria of the genus Bacillus form endospores that are extremely resistant to damage by heat, radiation, desiccation, and toxic chemicals (reviewed by Losick et al. (1986) Ann Rev Genet 20:625-669). This phenomenon is attributed to extensive intermolecular cross-linking of the coat proteins. In certain embodiments of the subject method, such as those which include relatively harsh affinity separation steps, Bacillus spores can be the preferred display package. Endospores from the genus Bacillus are more stable than are, for example, exospores from Streptomyces. Moreover, Bacillus subtilis forms spores in 4 to 6 hours, whereas Streptomyces species may require days or weeks to sporulate. In addition, genetic knowledge and manipulation is much more developed for B. subtilis than for other spore-forming bacteria.

Viable spores that differ only slightly from wild-type are produced in B. subtilis even if any one of four coat proteins is missing (Donovan et al. (1987) J. Mol. Biol. 196:1-10). Moreover, plasmid DNA is commonly included in spores, and plasmid encoded proteins have been observed on the surface of Bacillus spores (Debro et al. (1986) J. Bacteriol. 165:258-268). Thus, it can be possible during sporulation to express a gene encoding a chimeric coat protein comprising a polypeptide of the variegated gene library, without interfering materially with spore formation.

To illustrate, several polypeptide components of B. subtilis spore coat (Donovan et al. (1987) J. Mol. Biol. 196:1-10) have been characterized. The sequences of two complete coat proteins and amino-terminal fragments of two others have been determined. Fusion of the test polypeptide sequence to cotC or cotD fragments is likely to cause the polypeptide to appear on the spore surface. The genes of each of these spore coat proteins are preferred as neither cotC or cotD are post-translationally modified (see Ladner et al. U.S. Pat. No. 5,223,409).

iv) Selecting Peptides from the Display Mode

Upon expression, the variegated polypeptide display is subjected to affinity enrichment in order to select for test polypeptides which bind preselected lipid kinase inhibitors. The term “affinity separation” or “affinity enrichment” includes, but is not limited to: (1) affinity chromatography utilizing immobilized lipid kinase inhibitors, and (2) precipitation or pull-down experiments using soluble lipid kinase inhibitors. In each embodiment, the library of display packages are ultimately separated based on the ability of the associated test polypeptide to bind the lipid kinase inhibitor of interest. See, for example, the Ladner et al. U.S. Pat. No. 5,223,409; the Kang et al. International Publication No. WO 92/18619; the Dower et al. International Publication No. WO 91/17271; the Winter et al. International Publication WO 92/20791; the Markland et al. International Publication No. WO 92/15679; the Breitling et al. International Publication WO 93/01288; the McCafferty et al. International Publication No. WO 92/01047; the Garrard et al. International Publication No. WO 92/09690; and the Ladner et al. International Publication No. WO 90/02809. In most preferred embodiments, the display library will be pre-enriched for peptides specific for the lipid kinase inhibitor by first contacting the display library with any negative controls or other lipid kinase inhibitors for which differential binding by the test polypeptide is desired. Subsequently, the non-binding fraction from that pre-treatment step is contacted with the lipid kinase inhibitor and peptides from the display which are able to specifically bind the lipid kinase inhibitor are isolated.

With respect to affinity chromatography, it will be generally understood by those skilled in the art that a great number of chromatography techniques can be adapted for use in the present invention, ranging from column chromatography to batch elution, and including ELISA and biopanning techniques. Typically, where lipid kinase inhibitor is or can be immobilized on an insoluble carrier, such as sepharose or polyacrylamide beads, or, alternatively, the wells of a microtitre plate.

The population of display packages is applied to the affinity matrix under conditions compatible with the binding of the test polypeptide to the lipid kinase inhibitor. The population is then fractionated by washing with a solute that does not greatly effect specific binding of polypeptides to the lipid kinase inhibitor, but which substantially disrupts any non-specific binding of the display package to the lipid kinase inhibitor or matrix. A certain degree of control can be exerted over the binding characteristics of the polypeptides recovered from the display library by adjusting the conditions of the binding incubation and subsequent washing. The temperature, pH, ionic strength, divalent cation concentration, and the volume and duration of the washing can select for polypeptides within a particular range of affinity and specificity. Selection based on slow dissociation rate, which is usually predictive of high affinity, is a very practical route. This may be done either by continued incubation in the presence of a saturating amount of free lipid kinase inhibitor (if available), or by increasing the volume, number, and length of the washes. In each case, the rebinding of dissociated polypeptide-display package is prevented, and with increasing time, display packages of higher and higher affinity are recovered. Moreover, additional modifications of the binding and washing procedures may be applied to find polypeptides with special characteristics. The affinities of some peptides are dependent on ionic strength or cation concentration. This is a useful characteristic for peptides to be used in affinity purification of various proteins when gentle conditions for removing the protein from the peptide are required. Specific examples are polypeptides which depend on Ca⁺⁺ for lipid kinase inhibitor binding activity and which lose or gain binding affinity in the presence of EGTA or other metal chelating agent. Such peptides may be identified in the recombinant polypeptide library by a double screening technique isolating first those that bind the lipid kinase inhibitor in the presence of Ca⁺⁺, and by subsequently identifying those in this group that fail to bind in the presence of EGTA, or vice-versa.

After “washing” to remove non-specifically bound display packages, when desired, specifically bound display packages can be eluted by either specific desorption (using excess lipid kinase inhibitor) or non-specific desorption (using pH, polarity reducing agents, or chaotropic agents). In preferred embodiments, the elution protocol does not kill the organism used as the display package such that the enriched population of display packages can be further amplified by reproduction. The list of potential eluants includes salts (such as those in which one of the counter ions is Na⁺, NH₄⁺, Rb⁺, SO₄²⁻, H₂PO₄⁻, citrate, K⁺, Li⁺, Cs⁺, HSO₄⁻, CO₃²⁻, Ca²⁺, Sr²⁺, Cl⁻, PO₄²⁻, HCO₃⁻, Mg₂⁺, Ba₂⁺, Br⁻, HPO₄²⁻, or acetate), acid, heat, and, when available, soluble forms of the lipid kinase inhibitor. Because bacteria continue to metabolize during the affinity separation step and are generally more susceptible to damage by harsh conditions, the choice of buffer components (especially eluates) can be more restricted when the display package is a bacteria rather than for phage or spores. Neutral solutes, such as ethanol, acetone, ether, or urea, are examples of other agents useful for eluting the bound display packages.

In preferred embodiments, affinity enriched display packages are iteratively amplified and subjected to further rounds of affinity separation until enrichment of the desired binding activity is detected. In certain embodiments, the specifically bound display packages, especially bacterial cells, need not be eluted per se, but rather, the matrix bound display packages can be used directly to inoculate a suitable growth media for amplification.

Where the display package is a phage particle, the fusion protein generated with the coat protein can interfere substantially with the subsequent amplification of eluted phage particles, particularly in embodiments wherein the cpIII protein is used as the display anchor. Even though present in only one of the 5-6 tail fibers, some peptide constructs because of their size and/or sequence, may cause severe defects in the infectivity of their carrier phage. This causes a loss of phage from the population during reinfection and amplification following each cycle of panning. In one embodiment, the peptide can be derived on the surface of the display package so as to be susceptible to proteolytic cleavage which severs the covalent linkage of at least the target binding sites of the displayed peptide from the remaining package. For instance, where the cpIII coat protein of M13 is employed, such a strategy can be used to obtain infectious phage by treatment with an enzyme which cleaves between the test polypeptide portion and cpIII portion of a tail fiber fusion protein (e.g. such as the use of an enterokinase cleavage recognition sequence).

To further minimize problems associated with defective infectivity, DNA prepared from the eluted phage can be transformed into host cells by electroporation or well known chemical means. The cells are cultivated for a period of time sufficient for marker expression, and selection is applied as typically done for DNA transformation. The colonies are amplified, and phage harvested for a subsequent round(s) of panning.

After isolation of display packages which encode polypeptides having a desired binding specificity for the lipid kinase inhibitor, the test polypeptides for each of the purified display packages can be tested for biological activity in the secretion mode of the subject method.

(v) Generations of Polypeptide Libraries

The variegated polypeptide libraries of the subject method can be generated by any of a number of methods, and, though not limited by, preferably exploit recent trends in the preparation of chemical libraries. For instance, chemical synthesis of a degenerate gene sequence can be carried out in an automatic DNA synthesizer, and the synthetic genes then ligated into an appropriate expression vector. The purpose of a degenerate set of genes is to provide, in one mixture, all of the sequences encoding the desired set of potential test sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

As used herein, “variegated” refers to the fact that a population of peptides is characterized by having a peptide sequence which differ from one member of the library to the next. For example, in a given peptide library of n amino acids in length, the total number of different peptide sequences in the library is given by the product of {ν₁×ν₂× . . . νₙ₋₁×νₙ} where each νₙ represents the number different amino acid residues occurring at position n of the peptide. In a preferred embodiment of the present invention, the peptide display collectively produces a peptide library including at least 96 to 10⁷ different peptides, so that diverse peptides may be simultaneously assayed for the ability to interact with the lipid kinase inhibitor.

In one embodiment, the test polypeptide library is derived to express a combinatorial library of peptides which are not based on any known sequence, nor derived from cDNA. That is, the sequences of the library are largely, if not entirely, random. It will be evident that the peptides of the library may range in size from dipeptides to large proteins.

In another embodiment, the peptide library is derived to express a combinatorial library of peptides which are based at least in part on a known polypeptide sequence or a portion thereof (though preferably not a cDNA library). That is, the sequences of the library is semi-random, being derived by combinatorial mutagenesis of a known sequence(s). See, for example, Ladner et al. PCT publication WO 90/02909; Garrard et al., PCT publication WO 92/09690; Marks et al. (1992) J. Biol. Chem. 267:16007-16010; Griffths et al. (1993) EMBO J. 12:725-734; Clackson et al. (1991) Nature 352:624-628; and Barbas et al. (1992) PNAS 89:4457-4461. Accordingly, polypeptide(s) which are known binding partners for a lipid kinase inhibitor can be mutagenized by standard techniques to derive a variegated library of polypeptide sequences which can further be screened for agonists and/or antagonists. The purpose of screening such combinatorial peptide libraries is to generate, for example, homologs of known polypeptides which can act as either agonists or antagonists, or alternatively, possess novel activities all together. To illustrate, a ligand can be engineered by the present method to provide more efficient binding or specificity to a cognate receptor, yet still retain at least a portion of an activity associated with wild-type ligand. Thus, combinatorially-derived homologs can be generated to have an increased potency relative to a naturally occurring form of the protein. Likewise, homologs can be generated by the present approach to act as antagonists, in that they are able to mimic, for example, binding to the lipid kinase inhibitor, yet not induce any biological response, thereby inhibiting the action of authentic ligand.

In preferred embodiments, the combinatorial polypeptides are in the range of 3-100 amino acids in length, more preferably at least 5-50, and even more preferably at least 10, 13, 15, 20 or 25 amino acid residues in length. Preferably, the polypeptides of the library are of uniform length. It will be understood that the length of the combinatorial peptide does not reflect any extraneous sequences which may be present in order to facilitate expression, e.g., such as signal sequences or invariant portions of a fusion protein.

The harnessing of biological systems for the generation of polypeptide diversity is now a well established technique which can be exploited to generate the peptide libraries of the subject method. The source of diversity is the combinatorial chemical synthesis of mixtures of oligonucleotides. Oligonucleotide synthesis is a well-characterized chemistry that allows tight control of the composition of the mixtures created. Degenerate DNA sequences produced are subsequently placed into an appropriate genetic context for expression as polypeptides.

There are two principal ways in which to prepare the required degenerate mixture. In one method, the DNAs are synthesized a base at a time. When variation is desired at a base position dictated by the genetic code a suitable mixture of nucleotides is reacted with the nascent DNA, rather than the pure nucleotide reagent of conventional polynucleotide synthesis. The second method provides more exact control over the amino acid variation. First, trinucleotide reagents are prepared, each trinucleotide being a codon of one (and only one) of the amino acids to be featured in the polypeptide library. When a particular variable residue is to be synthesized, a mixture is made of the appropriate trinucleotides and reacted with the nascent DNA. Once the necessary “degenerate” DNA is complete, it must be joined with the DNA sequences necessary to assure the expression of the polypeptide, as discussed in more detail below, and the complete DNA construct must be introduced into the cell.

Whatever the method may be for generating diversity at the codon level, chemical synthesis of a degenerate gene sequence can be carried out in an automatic DNA synthesizer, and the synthetic genes can then be ligated into an appropriate gene for expression. The purpose of a degenerate set of genes is to provide, in one mixture, all of the sequences encoding the desired set of potential test polypolypeptide sequences. The synthesis of degenerate oligonucleotides is well known in the art (see for example, Narang, S A (1983) Tetrahedron 39:3; Itakura et al. (1981) Recombinant DNA, Proc 3rd Cleveland Sympos. Macromolecules, ed. A G Walton, Amsterdam: Elsevier pp273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, for example, Scott et al. (1990) Science 249:386-390; Roberts et al. (1992) PNAS 89:2429-2433; Devlin et al. (1990) Science 249: 404-406; Cwirla et al. (1990) PNAS 87: 6378-6382; as well as U.S. Pat. Nos. 5,223,409, 5,198,346, and 5,096,815).

Experimental Procedures

A) Microbiology Techniques

i) Growing and Storing of E. Coli Strains

Cultures of E. coli were grown overnight at 37° C. with shaking. E. coli containing plasmids were selected on LB-plates by adding an appropriate antibiotic for which a resistance gene is encoded in the plasmid. E. coli strains were stored as glycerol cultures (20% glycerol, 80% LB-medium) at −80° C.

ii) Preparation of Transformation Competent Cells

Electrocompetent cells (BJ5183): A 10 ml LB starter culture containing 30 μg/ml streptomycin was inoculated with a freshly grown BJ5183 colony and grown overnight with shaking at 37° C. 2 ml of the starter culture were diluted into 11 of LB medium with 30 μg/ml streptomycin and grown with shaking at 37° C. When an OD₅₅₀ of about 0.8 was reached, the cells were collected and incubated for 45 min on ice. The cells were pelleted by centrifugation at 2600 g for 10 min at 4° C. The pellet was washed by resuspension in 11 sterilized, ice-cold 10% glycerol and an additional spin at 2500 g for 30 min performed. The washing steps were repeated and all but 2 ml of the supernatant was discarded. The remaining 2 ml were used to resuspend the pellet, and then 40 μl aliquots of the cells were snap frozen in liquid nitrogen and stored at −80° C.

Chemically competent cells (JM110, DH5α): Most strains prepared by the RbCl methods have a higher transformation efficiency than cells prepared by the CaCl₂ procedure. 4 ml of LB-medium were inoculated with a single colony and grown overnight as a starter culture. 3 ml of the starter culture was diluted into 500 ml LB-medium containing 20 mM MgSO₄ and grown at 37° C. When an OD₅₉₀ of 0.5-0.6 was reached, the cells were harvested by centrifugation for 5 min at 500 g. The pellet was gently resuspended in 200 ml TFBI and incubated on ice for 5 min. The cells were pelleted as before and resuspended in 10 ml TFBII. After incubating on ice for 60 min, the cells were frozen as 100 μl aliquots in liquid nitrogen and stored at −80° C.

B) Molecular Biology Techniques

i) Transformation

Chemical transformation: 50 μl of competent cells were thawed on ice and immediately incubated with 1-3 ng plasmid DNA on ice for 30 minutes. The sample was heat-shocked at 42° C. for 45 sec, followed by another incubation for 2 min on ice. LB-medium was added (1 ml) and the culture was incubated at 37° C. with shaking for 20 min. Afterwards, the cells were centrifuged at 3500 g for 5 min and all but ˜100 μl of the LB-medium was removed. The bacteria were resuspended in the remaining LB-medium and plated on LB-plates with the desired antibiotic and incubated overnight at 37° C.

ii) Preparation of Plasmid DNA

Preparation with the Maxi-Plasmid preparation kit (Qiagen): The manufacturer's suggested protocol was modified for use of 500 ml medium instead of the recommended 250 ml to grow the cells. For large plasmids (>25 kb), a few additional changes were made. The volume of the buffers PI, PII and PIII were doubled and the elution buffer was heated to 65° C. prior to elution from the column.

Preparation with the Mini-preparation Kit (Qiagen): Mini-preparations of DNA were carried out according to the manufacturer's protocol.

Alkaline Lysis Mini-preparation: 1.5 ml of E. coli culture grown overnight was pelleted by centrifuging at 3500 g for 5 minutes. The supernatant was discarded and the pellet was resuspended in 200 μl of resuspension buffer. To lyse, 200 μl of lysis solution was added, then the tube was gently mixed and incubated for 5 min at RT. By adding 200 μl of precipitation solution, a precipitate was formed that contained chromosomal DNA and some part of RNA. The tubes were spun at 14,000 g for 5 minutes and the supernatant was recovered and placed in a fresh tube. The DNA was precipitated by adding 500 μl isopropanol and spun for 5 min at 14,000 g. Then, the pellet then was washed in 500 μl 70% ethanol, centrifuged under the previous conditions and the supernatant was discarded. After the pellet was air-dried, it was dissolved in 50 μl water.

iii) Polymerase Chain Reaction (PCR)

PCR was used to amplify and/or modify a fragment from of a DNA template. PCR was carried out in a solution containing 1/10 Vol reaction buffer, 1 μM of each primer (forward and reverse), 0.5 μM of each dNTP, 1 μl DNA-template (less than 0.5 μg), 3 μl MgSO₄, 0.5 μl Taq-HiFi polymerase and water to a final volume of 50 μl.

The reaction was carried out in a PCR thermo cycler for 25 cycles. The following program was used: denaturation of the template at 94° C. for 5 min in the first cycle and then 25 cycles of denaturation for 30 sec at 30° C., annealing for 30 sec at 55° C., and synthesis for 1.5 min at 68° C. At the end, a final extension step was added for completion of the polymerase reaction at 68° C. for 5 min.

vi) Restriction Digests

Restriction digests of plasmid DNA were carried out in a total volume of 50-100 μl. Each reaction included 1/10 volume of 10×reaction buffer, plasmid DNA, 1/10 volume of 10×BSA if required for the enzyme used and 5-15 U of restriction enzyme (New England Biolabs). This reaction mixture was incubated for 1-3.5 hours at 37° C. When the DNA was simultaneously digested by two restriction enzymes, the buffer which gave the highest level of activity for both enzymes was chosen.

v) Dephosphorylation of DNA-Fragments

Calf intestinal alkaline phosphatase (CIAP) (Invitrogen) dephosphorylates phosphate groups at the 5′ end of DNA preventing self-ligation of a plasmid with cohesive or blund ends. The reaction was carried out in a total volume of 100 μl which contained the digested DNA, 5 μl of 10×NEIII buffer and calf intestinal alkaline phosphatase (5-10 U for 100 μg vector DNA). The samples were incubated at 37° C. for 90 min.

vi) Subcloning into pGem-T Vector

The pGem-T system (Promega) contains the vector pGem-T which has a T-overhang. This allows direct insertion of PCR products which are produced with an overhanging adenine by Taq polymerase.

For the pGem-T system, a typical ligation mix consisted of 5 μl of 2×rapid ligation buffer provided with the kit, 1 μl pGem-T vector, 1 μl T4 DNA ligase, different amounts of insert (gel purified PCR product) and water to a final volume of 10 μl. Typical vector to insert molar ratios were 1:3-3:1. The reaction was incubated overnight at 4° C. The next day, the ligation products were transformed into competent cells, and plated on LB Amp plates treated with 100 μl of 0.5 mM IPTG and 20 μl of 50 mg/ml X-Gal on the surface (incubated for 30 min at 37° C. prior to use to absorb the liquid) and incubated overnight.

pGem-T is a vector that allows blue and white screening as successful cloning of an insert interrupts the coding sequence of β-galactosidase. Recombinant clones can be screened by color. Clones that contain the insert should, in most cases produce white colonies. Blue colonies might still contain the insert if it was cloned in frame with the lacZ gene and does not contain stop codons. Only the white colonies were picked and analyzed by restriction digest.

vii) Ligation

Ligation reactions were usually carried out in a volume of 10 μl, containing 1 μl of 10 mM ATP, 1 μl of ligase, 1 μl of 10×ligase reaction buffer and various amounts of vector and insert. Ratios from 1:3 to 3:1 of insert to vector were used. The ligation reaction was incubated at 4° C. for 2 hours.

viii) DNA Agarose Electrophoresis

Agarose electrophoresis is used to determine the size of or to separate DNA. 100 ml of TAE-agarose solution was boiled in the microwave containing agarose concentrations ranging from 0.8-1.2%. After the agarose solution cooled down to about 50° C., 2 μl of 10 mg/ml stock ethidium bromide solution was added and the gel was poured. The gel was allowed to harden at RT and placed into a gel running chamber in TAE-buffer. 6×sample buffer was added to the samples before loading. The gel was usually run at 80 V for 1-2.5 hours and the DNA was detected under UV light by the fluorescence dye ethidium bromide which intercalates in the DNA.

ix) Gel Extraction

After DNA bands were separated by electrophoresis, it is possible to purify bands of interest for ligation or sequencing reactions. Under the UV light, the bands were quickly excised from of the agarose gels to prevent the formation of pyrimidine dimers. The extraction was carried out with the QIAEXII gel extraction kit (Qiagen) according to the manufacturer's protocol.

x) Phenol/Chloroform Extraction

An equal volume of phenol:chloroform:isoamylalcohol (25:24:1) was added to the nucleic acid sample and the contents mixed until an emulsion was formed. This mixture was centrifuged at 14,000 g for 1 min and the aqueous phase was transferred to a fresh tube. The phenol:chloroform:isoamylalkohol mixture was reextracted with an equal volume of water and the aqueous phases were then combined. An equal volume of chloroform was added to the aqueous phase and the tube was then vortexed and centrifuged. DNA in the aqueous phase was precipitated by adding 1/10 Vol of 3 M sodium acetate followed by 2-2.5 Vol of ice-cold ethanol. This mixture was incubated for 15 min on ice, followed by a centrifugation step at 14,000 g for 10 min at 4° C. The supernatant was removed by aspiration and the pellet was washed in 500 μl 70% ethanol and centrifuged again at 14,000 g for 10 min at 4° C. The supernatant was removed, and after air-drying the pellet, the DNA sample was dissolved in the desired volume of water.

xi) Determining the Concentration of DNA

The concentration of DNA solutions were measured spectrophotometrically. One absorbance unit at 260 nm (OD₂₆₀) correlates to 50 μg of double-stranded DNA in 1 ml of solution in a cuvette with 1 cm pathlength.

The ratio of absorbance at 260 and 280 nm is used to determine the purity of nucleic acids. Pure DNA has a ratio of 1.8-1.9. Lower values indicate protein or phenol contamination while higher values are due to RNA.

C) Tissue Culture

i) Culturing Cells

Cells were grown in the appropriate media at 37° C. in the presence of 5% CO₂. Every 2-3 days, the medium was either changed or the cells were passaged, when about 90% confluency was reached. The cells were split in a range between 1:3 to 1:10. Splitting of cells was performed by aspiration of the media, washing with PBS, addition of 1 ml trypsin/EDTA (2 min incubation), then redistribution of the detached cells after resuspension in fresh medium by pipetting to new dishes. 10 ml of media was used for one 10 cm dish.

ii) Storing Tissue Culture Cells

Cells were harvested and collected by a 5 min spin at 500 g. The pellet was resuspended in freezing media and the cells slowly frozen to −80° C. After 2 days the cells were placed in the liquid nitrogen tank for long term storage.

An aliquot of cells was thawed by incubation at 37° C. Then, the cells were directly added to a 10 cm dish with 10 ml medium. After ˜5 hours, the medium was changed to remove the DMSO in the freezing media.

iii) Harvesting Cells

The media was aspirated off and the cells were washed with 5 ml of 1×PBS. 3 ml 1×PBS containing 0.5 mM ETDA was added to lift the cells prior to pipetting up and down. The cells were then washed twice in PBS. The spins were carried out at 1000 g for 10 min.

iv) Cell Counting

10 μl of cell suspension and 10 μl of trypane blue were mixed and 10 μl of this mixture were loaded on a hemacytometer. The cells in the four corner squares were counted using the microscope. The average number of cells in one square was multiplied by 20,000 which resulted in the number of cells per ml.

v) Incubation with Wortmannin/Biotin-Wortmannin

Tissue culture cells were incubated with 40-50 μM wortmannin/biotin-wortmannin in serum free medium. The media and the inhibitory compound were premixed to reduce toxicity and incubated on the cells for 45 min or 1 h.

D) Biochemical and Immunological Methods

i) Nuclear Extract Preparation

Cells were pelleted by spinning at 600 g for 5 min and washed with 30×pellet volume of cold PBS. After spinning at 600 g for 5 min, the cells were resuspended in 5×pellet volume of buffer A and immediately centrifuged as above. To lyse the cells, they were resuspended in 2 pellet volumes of buffer A (+0.5 mM DTT, +0.5 mM PMSF), transferred to a dounce-homogenizer and gently lysed with approximately 40 strokes. Intact nuclei were monitored by standard microscopy. As soon as the cells were lysed, the lysate was centrifuged at 1000 g for 30 min and the supernatant (cytosolic fraction) removed. The pellet which contained the nuclei, was resuspended in 3 ml of buffer B (+0.5 mM DTT, +0.5 mM PMSF) for every 10⁹ cells originally used. The nuclear suspension was transferred to the dounce-homogenizer and lysed with about 20 strokes. The homogenate was transferred to a beaker and stirred for 30 min at 4° C. To clarify the solution, a centrifugation step at 14,000 g for 10 min was added and the supernatant was stored as the nuclear fraction at −80° C.

ii) Cell Lysis

Cells from one plate (10 cm) were pelleted and lysed in 100 μl of TGN lysis buffer containing protease inhibitors for 20 min at 4° C. To clarify the supernatant, the lysate was centrifugated at 14,000 g for 10 minutes at 4° C.

iii) Measurement of Protein Concentrations

The protein concentration of various samples was determined by comparison to a BSA standard curve which was always prepared. The Biorad protein solution was diluted fivefold into water. 1 ml of this solution was added in a 1 cm plastic cuvette. To this solution, the desired amount of BSA or 1-5 μl of the sample was added. After mixing and incubating for 5 min, the OD₅₉₅ was measured and the concentration determined relative to the BSA standard curves.

iv) Anti-Flag Immunoprecipitation

Preclear: For five 10 cm plates of cells, 30 μl of Mouse IgG and 120 μl of Protein A Agarose beads were precomplexed in PBS overnight. Thereupon, they were washed 3 times in TGN buffer and used to preclear the lysate for 1 hour at 4° C.

Immunoprecipitation: Prior to use, the Flag beads were washed once in 350 mM glycine, pH 3.5 and 3×in TGN buffer. 30 μl of M2 Flag beads were used for every reaction (˜½ a plate of cells) and the immunoprecipitation was carried out for 2 hours at 4° C.

Washes: Each reaction was washed 3×with 300 μl TGN buffer, 2×with 500 μl LiCl buffer and 2×with kinase buffer.

Elution: Flag epitope-tagged proteins were recovered from the Flag affinity resin by rotating the resin twice for 15 min with 2 resin volumes of elution buffer at RT.

v) BW Incubations

A TR kinase assay: The beads for one reaction were incubated with 100 μl kinase buffer containing the desired wortmannin/biotin-wortmannin concentration for 15 min at 30° C. After the incubation, the beads were washed in 500 μl kinase buffer.

DNA-PK kinase assay: The wortmannin/biotin-wortmannin incubation preceding the DNA-PK kinase assay was carried out in a volume of 30 μl containing 2 μl of EcoRI digested pBJFATR plasmid (100 ng/μl), 18.5 μl buffer B, 7.5 μl H₂O, 0.5 μl purified DNA-PK (Promega) and 1.5 μl of 1:10 dilutions of wortmannin-biotin wortmannin compounds in water. This mixture was incubated for 10 minutes at RT.

Cell extract: Cell extract was precleared with Streptavidin beads and the incubation with wortmannin/biotin-wortmannin was carried out in a total volume of 50 or 100 μl. Typically, the protein concentration was between 1-1.5 μg/μl in T7.5 buffer containing 1 mM DTT and wortmannin/biotin-wortmannin concentrations from 0.5-50 μM. The mixture was incubated for 15 min at 4° C. or 30° C.

Competition: For competition experiments, nuclear extract was pretreated with either 150 μM wortmannin at 30° C. for 10 min or heated at 95° C. for 10 min prior to the biotin-wortmannin incubation at 30° C. for 15 min. For the competition with ATP, ATP was directly added to a final concentration of 1 mM ATP.

vi) Streptavidin-Sepharose Pull-Down

Prior to use, the streptavidin beads were washed 4×in T7.5 buffer. The third wash also contained BSA (100 μg/ml) to saturate unspecific protein interactions.

Lysate previously incubated with wortmannin/biotin-wortmannin was added to 50 μl slurry of streptavidin sepharose beads and buffer was added to a minimum reaction volume of 100 μl. The mixture was incubated at 4° C. for 10-20 min, then the beads were washed twice in 500 μl wash buffer.

vii) Kinase Assays

ATR kinase assay: ATR precipitates were incubated with 27.5 μl of kinase buffer+1 μl 32P γ-ATP (1 μCi)+1.5 μl GST-RAD17 substrate (1.5 μg) for exactly 15 min at 30° C. The reactions were stopped by adding 10 μl of 6×SDS sample buffer and boiling the sample for 8 min at 95° C.

DNA-PK kinase assay: To the 30 μl mixture resulting from the wortmannin/bitoin-wortmannin incubation, 0.3 μl 32P γ-ATP, 0.8 μl of 10 mM ATP, 2 μl substrate (1 μg/μl), 4 μl buffer A and 3 μl buffer B were added and the mixture incubated for 15 min at 30° C. The kinase reaction was stopped by adding 10 μl of 6×SDS sample buffer and boiling for 8 min at 95° C.

viii) SDS Polyacrylamide Gel Electrophoresis

SDS denaturing gel electrophoresis was used to separate proteins according to their size. The Laemmli buffer system with glycine as a zwitterion was used. The electrophoresis was carried out vertically in gels of either 0.75 or 1.5 mm thickness. On top of the resolving gel, a stacking gel of ˜2 cm was poured.

The following table shows the recipes for 10 ml of 5%, 12.5% and 15% resolving gels and 7 ml of a 4% stacking gel. For minigels, the volume indicated in one column of Table 1 is sufficient. For large gels, 2×the recipe was used. The ingredients were premixed and the polymerization reaction was started by adding APS.

TABLE 1
Recipes for different percentage resolving gels and the stacking gel for
SDS-PAGE.

| | 5% | 12.5% | 15% | Stacking |
|---|---|---|---|---|
| Acrylamide (ml) | 1.7 | 4.2 | 5 | 1 |
| Water (ml) | 5.8 | 3.2 | 2.4 | 4.25 |
| 10% SDS (μl) | 100 | 100 | 100 | 60 |
| Resolving buffer (ml) | 2.5 | 2.5 | 2.5 | — |
| Stacking buffer (ml) | — | — | — | 0.75 |
| TEMED (μl) | 10 | 10 | 10 | 7 |
| APS (μl) | 50 | 50 | 50 | 30 |

Preparation of the samples. An adequate volume of 6×SDS sample buffer was added to the samples. The samples were then heated for 5-10 min at 95° C., quickly centrifuged and loaded onto an SDS-PAGE gel. Gels were run at 100 V until the samples entered the stacking gel and then at 150-200 V until the dye front reached the bottom of the gel.

Gradient gels. For gradient gels, two different concentrated acrylamide solutions were prepared and to the higher percentage solution, 1.5 g of sucrose was added. Using a gradient mixer, a gradient between the two solutions was generated and the gels were either poured from top (big gels) or slowly from the bottom in a multicaster (minigels).

E) Detection Methods

i) Coomassie Stain

The staining solution (45% methanol, 10% acetic acid, 0.25 g Coomassie brilliant blue in 100 ml) was heated for 10 sec in the microwave prior to staining the gel for 1 hour. The gel was destained in destain solution (45% methanol, 10% acetic acid) for about 4-10 hours during which the destain solution was changed ˜4 times.

ii) Silver Stain

Silver staining was used when a more sensitive detection method than Coomassie was required. The gel was fixed overnight in the Fix solution. The next morning it was washed three times in 50% methanol for 15 min per wash. The gel was pretreated with 0.02% Na₂S₂O₃ for one minute and directly rinsed 3×20 sec in distilled water. For the next 20 minutes, the gel was impregnated in the silver nitrate containing impregnate solution for 20 min and then rinsed two times for 30 sec in distilled water. To develop the gel, developing solution was added and incubated until the level of detection was adequate. The staining process was stopped by adding stop solution for 10 min. For further storage or in preparation for drying, the gel was washed for at least 20 min in 50% methanol.

iii) Drying Gels

Gels were washed in a solution containing 3% glycerol, 20% methanol for 45 min and then transferred to Whatman paper, covered with Saran wrap and placed on the gel dryer. Under vacuum, the gel was dried for 1.5 h at 80° C.

iv) Autoradiography

The dried gel was either exposed overnight to Biomax film or in the phosphoimager cassette for several hours and quantified using the program Image Quant v1.2 from Molecular Dynamics, Sunnyvale, Calif. (USA). Curves were plotted in the program KaleidaGraph v3.5 (Synergy Software, Reading, Pa.) using the Hill equation.

v) Transfer to PVDF Membrane

In order to analyze proteins from a gel using immunohistochemical methods, the proteins were transferred from SDS-PAGE gels to PVDF membrane. Therefore, the membrane was preincubated for 10 min each in methanol, distilled water and transfer buffer. For the transfer, a “sandwich” consisting of Whatman paper, gel (without the stacking gel), membrane and Whatman paper was built and the transfer was performed in a blotting chamber in transfer buffer at either 360 mA for 3 hours for a minigel or 1000 mA for 3 hours for a large gel.

vi) Developing Western Blots

After the transfer, the membrane was blocked in PBS-T containing 5% dry milk for 45 min, washed in PBS-T and incubated for the indicated time (Table 2) with the desired antibody. Before the 30 min incubation with the secondary antibody (α-mouse/α-rabbit) conjugated to horseraddish peroxidase (HRP), the membrane was washed 3 times in PBS-T. A final series of washes was performed prior to analyzing the blot. The blot was developed using Supersignal (Pierce) according to the manufacturer's recommendations. This was comprised of using equal volumes of each solution and incubating for 5 minutes. The signal was detected according to the principle of chemiluminescence on X-Omat-Blue film. If the signal was used to correct for equal loading, the signal was quantified using the program NIH Image v1.62 (Research Services Branch —NIH).

TABLE 2
Overview over the conditions used for the incubation with different
primary and secondary antibodies.

| | | | Incubation | |
|---|---|---|---|---|
| Name | Dilution | Buffer | Time | Organism |
| α-ATM | — | — | 2 h | Mouse |
| α-ATR (1163) | 1:1000 | PBS-T, 5% dry | 1 h | Rabbit |
| | | milk | | |
| α-DNA PK_{cs} | 1:1000 | PBS-T, | 1 h | Rabbit |
| α-Flag M5 | 1:1000 | PBS-T | 1 h | Mouse |
| α-Wortmannin | 1:2000 | PBS-T | 1 h | Rabbit |
| Streptavidin- | 1:2000 | PBS-T | 30 min | |
| HRP | | | | |
| Goat α-Mouse | 1:5000 | PBS-T | 30 min | Goat |
| Goat α-Rabbit | 1:5000 | PBS-T | 30 min | Goat |

vii) Stripping Blots

Before reuse of a blot with another primary antibody, a blot was stripped to disrupt interactions between antibodies and their recognized epitopes on membranes. Therefore, the membrane was incubated in stripping buffer for 10-15 min in a 50° C. waterbath and gently shaken every 5 min. After applying the stripping solution, the membrane was washed three times in PBS-T to remove residual stripping solution.

F) Chemical Methods

i) Thin Layer Chromatography

For thin layer chromatography (TLC), Silica gel glass plates (0.25 mm) were used to separate different components in hexane:ethylacetate (1:2) as solvents. Components were visualized by illumination with long wave ultraviolet light and by dipping into an aqueous solution of ceric ammonium molybdate followed by heating.

### EXAMPLE 1

### Characterization of a Bifunctional Wortmannin Derivative

i) Generation of Biotin-Wortmannin Derivatives

We took advantage of previous structure-function studies to determine where to link wortmannin to biotin.

11-O-desacetyl wortmannin is only 4-fold less effective than wortmannin towards PI 3-kinase (IC₅₀ of 16.7 nM for the 11-O-desacetyl compound versus 4.2 nM for wortmannin).

In addition, modification of the hydroxy group at C11 with several different ester moieties did not significantly affect the activity to PI 3-kinase. Therefore, wortmannin was coupled to biotin via the C11 hydroxy group and a water-soluble. This suggested that the modification at C11 might not result in a significant loss of biological activity.

Two different constructs were synthesized. The biotinylated wortmannin derivative (BW) contains a PEG linker via which biotin is linked to wortmannin. A control compound (BC) was also prepared in which the wortmannin moiety was linked via a PEG linker to a methyl group.

ii) Stability of Wortmannin and its Derivatives

Thin layer chromatography (TLC) was used to determine the stability of wortmannin and biotin-wortmannin in different buffer systems at different temperatures. Tris based (pH 7.5, 8)and Hepes (pH 7.5, 8) based buffer systems were tested for incubations for 30 min at 4° C. and 30° C. The separation between these forms was possible because the ring-opened form which also might react with amines of wortmannin has a lower mobility (R_{F}˜0) in hexane:ethylacetate (1:2) than the active form which contains an intact furan ring with results in an RF value of about 0.47.

TABLE 3
Stability of wortmannin in different buffer. Using TLC, two different
forms can be detected of which one represents the active form (R_{F}˜0.47)
and the other the ring opened, inactive form (R_{F}˜0).

| | | Incubation | | |
|---|---|---|---|---|
| Buffer base | pH | temperature | R_{F}˜0 | R_{F}˜0.47 |
| Tris (20 mM) | 7.5 | 4° C. | − | + |
| Tris (20 mM) | 7.5 | 30° C. | − | + |
| Tris (20 mM) | 8 | 4° C. | − | + |
| Tris (20 mM) | 8 | 30° C. | − | + |
| Tris (1 M) | 7.5 | 30° C. | + | − |
| HEPES (20 mM) | 7.5 | 4° C. | − | + |
| HEPES (20 mM) | 7.5 | 30° C. | − | + |
| HEPES (20 mM) | 8 | 4° C. | − | + |
| HEPES (20 mM) | 8 | 30° C. | − | + |

This experiment showed that wortmannin was in large part stable at most conditions for at least 30 min. One exception was the incubation in 1 M Tris based buffer after which only the low mobility form could be detected. This experiment indicates that wortmannin, and because of the analogy probably also biotin-wortmannin, is stable at the conditions that were usually used for the reactions in the following experiments.

iii) Comparison of Cellular Targets of Wortmannin and Biotin-Wortmannin Derivatives

Lysate prepared from HEK 293T cells was precleared with streptavidin beads to remove endogenous streptavidin binding proteins. Subsequentley, the lysate was incubated with different concentrations of wortmannin or biotin-wortmannin ranging from 0.1 μM to 10 μM. Cellular proteins were separated by SDS gel electrophoresis, transferred to PVDF membrane, treated with α-wortmannin antiserum or streptavidin-HRP, respectively and visualized with chemiluminescence.

In general, the detected pattern of proteins that react with the two compounds was similar although one or two extra low molecular weight band were detected using the biotin-wortmannin compound starting at as low concentrations as 0.5 μM. The number of bands detected at each concentration was comparable with both inhibitors. A predominant high molecular weight band was observed at concentrations as low as 0.1 μM, although the intensity of the signal increases at 0.5 μM. At 2 μM, few new bands were detected but at 10 μM there was a strong increase in the intensity and number of bands and also differences between wortmannin and biotin-wortmannin. This suggested that the modification with the linker and biotin had only a small impact on the substrate specificity compared to wortmannin and that it might still specifically target members of the PI 3-kinase superfamily.

iv) Inhibition of ATR and DNA-PK Kinase Activity In Vitro by Wortmannin and its Derivatives

The ability of BW to react with PIK-related kinases in vitro through their kinase domain was investigated. Binding of wortmannin at the active site of these kinases inhibits their kinase activity. Kinase assays for ATR and DNA-PK, two members of the PIK-related kinase family, were carried out with increasing concentrations of wortmannin or biotin-wortmannin.

For ATR kinase assays, cells transfected with Flag-tagged ATR were harvested and lysates were prepared. Anti Flag immunoprecipitation was performed to obtain relatively pure ATR. GST-RAD 17 was used as a substrate and biotin-wortmannin/wortmannin concentrations ranging from 0.5 μM up to 50 μM or 100 μM were used.

DNA-PK kinase assays were carried out with purified DNA-PK. In this case, p53 was used as a substrate and wortmannin/biotin-wortmannin concentrations from 50 nm up to 5 μM were applied.

After the kinase reactions were carried out, the samples were separated on a 5-12.5% gradient gel. The gel was cut in half and the upper part (>80 kDa) was transferred to PVDF membrane. Flag-ATR and labeling with biotin-wortmannin were detected by chemiluminescence using α-Flag M5 or streptavidin HRP antibody. The lower half of the gel was stained with coomassie, dried and analyzed by autoradiography.

Analysis of substrate phosphorylation by autoradiography allows evaluation of the kinase activity that remains after incubation with different amounts of biotin-wortmannin. In the control lane, where no biotin-wortmannin was added, a corresponding amount of DMSO was added to exclude the possibility that some inhibition in this assay might be caused by DMSO. In the last lane, untransfected cells were used to determine the background substrate phosphorylation. The signal GST-Rad17 phosphorylation signal decreases significantly starting at biotin-wortmannin concentrations of 20-35 μM. The signal detected by Western analysis using streptavidin HRP shows that biotin-wortmannin binds to the kinase, increasingly with the biotin-wortmannin concentration added starting at 2 μM. In the lower lane, the amount of Flag-tagged ATR was determined by using a Flag specific antibody. This signal was used to normalize for equal loading to generate quantitative data.

Similar results were obtained after a DNA-PK kinase assay. The detected substrate phosphorylation shows a major decrease at biotin-wortmannin concentrations starting at 2 μM. The substrate coomassie stain also shows effective inhibition as lower mobility band (probably phosphorylated p53) just above unphosphorylated p53 vanishes at biotin-wortmannin concentrations starting at 1 μM. Detection of the compound biotin-wortmannin using streptavidin HRP results in a signal staring at 1 μM, saturating at 2 μM indicating that at these concentration a significant amount of biotin-wortmannin is bound to DNA-PKcs.

The reactivity of biotin-wortmannin was compared to the reactivity of wortmannin by determining the inhibitory concentration (50%) (IC50) of each compound for DNA-PK and ATR. Therefore in total, 3-4 experiments were carried out in which the substrate phosphorylation was quantified. For the ATR kinase assay, these values were corrected for equal loading by taking the anti-Flag Western signal as a reference.

Biotin-wortmannin was less sensitive than wortmannin for both ATR and DNA-PK as the substrate phosphorylation activity for biotin-wortmannin dropped slower than the substrate phosphorylation activity for wortmannin and resulted in flatter curves than the ones received for wortmannin. The inhibitory concentrations for 50% activity (IC₅₀) were determined from a graph. For ATR an IC₅₀ of 1.8 μM for wortmannin compared to an IC₅₀ of 10.8 μM for biotin-wortmannin was determined. For DNA-PK, the data resulted in an IC₅₀ of 0.14 μM for wortmannin compared to an IC₅₀ of 1.15 μM for biotin-wortmannin. Generally, this indicated that the modification of wortmannin resulted in an 6-8 fold loss of activity.

In vitro, biotin-wortmannin recognizes the same pattern of proteins as unmodified wortmannin which suggests that this compound is still able to specifically label members of the PI 3-kinase superfamily. Further, as the kinase activity inhibition of two members of the PIK-related kinases family could be detected, the implication can be made that it probably binds in a similar way in the active center mediated by a nucleophilic addition and that the linker does, in general, not interfere with this reaction. However, the inhibitory power of the biotin-wortmannin compound seems to be reduced. A comparison of the IC₅₀s of wortmannin and biotin-wortmannin for ATR, 1.8 μM and 10.6 μM respectively, showed an about 6 fold reduction in potency. Similar results were obtained for the IC₅₀s concerning DNA-PK. Wortmannin inhibits DNA-PK with an IC₅₀ of about 140 nM and biotin-wortmannin with and IC₅₀ of about 1.15 nm which results in an about 8 fold reduction in activity.

The detected IC₅₀S of wortmannin towards ATR and DNA-PK with 1.8 μM and 140 nM are within a reasonable range of the previously measured IC₅₀s with 1.8 μM for ATR and 16 nM for DNA-PK (Sarkaria et al., 1998). The higher discrepancy for the values for DNA-PK might be caused by the fact that IC₅₀s are not kinetic exact values as they vary for example for different amounts of protein kinase but still give a good idea of an inhibitor, especially when different ones are compared.

The 6-8 times less inhibitory potency of biotin-wortmannin compared to wortmannin is not further surprising as Norman et al. showed an about four fold reduction in activity for the 0-11-desacetylwortmannin derivative (Norman et al., 1996) and our compound was further chemically modified at the C11 by adding a PEG linker and Biotin. It was also expected, that the linker and Biotin might sterically inhibit the activity as unmodified wortmannin closely fits in the binding pocket thereby inducing a conformational change at the catalytic domain of porcine PI 3-kinase (Walker et al., 1996). The lower activity of the modified compound might also result from possible interactions of the linker or the biotin moiety with other proteins and therefore reducing the free, effective concentration.

v) Capability of the Wortmannin Derivatives to Enter Intact Cells

To determine if the biotinylated wortmannin compound can enter into live cells, HEK 293T cells (80% confluent) were incubated with either 50 μM wortmannin (W), 50 μM biotin-wortmannin (BW) or the corresponding amount of DMSO. After 45 min, the cells were harvested and then lysates were generated. 100 μg of protein was separated on a 5-12.5% gradient gel, transferred to PVDF membrane and either analyzed with α-wortmannin sera or with streptavidin HRP to detect biotin-wortmannin.

α-wortmannin sera recognized several proteins in extracts treated with wortmannin. However, no proteins were detected in the BW and in the control (DMSO) lanes. In contrast, using HRP for detection, no specific proteins were detected in the lane with extract gained from cells previously treated with biotin-wortmannin. Three background bands (endogenous streptavidin binding proteins) were observed in all three lanes. These results indicate that wortmannin is capable to enter into intact cells but biotin-wortmannin, however is not.

To further confirm this result, a second assay was performed in which plates of HEK 293T cells were transfected with Flag-tagged ATM. After 48 hours, the cells were incubated with DMSO as a control, 50 μM wortmannin (W) or 50 μM biotin-wortmannin (BW) for 1 hour at 37° C. The cells were harvested, lysed and an anti-Flag immunoprecipitation was performed. With these immunoprecipitates, a kinase assay was performed, using p53 as a substrate. Proteins were analyzed on a 5-15% gradient gel and the upper half of the gel was transferred to PVDF membrane and detected with α-Flag M5 antibody. The lower half of the gel was stained with coomassie and analyzed by P-32 autorad.

A reduction in the kinase activity measured by substrate phosphorylation is only observed in the cells previously incubated with wortmannin but not in cells treated with biotin-wortmannin or DMSO. Substrate phosphorylation which also leads to a decrease in the mobility of p53 as shown by coomassie stain, was measured by autoradiography. In the control lane (CTR) lysate from untransfected cells was analyzed to determine background substrate phosphorylation.

The data was quantified using the signal detected from anti-Flag Western detection to correct for equal levels of the ATM kinase. Normalizing of the ATM substrate phosphorylation signal detected from cells previously incubated with DMSO to 1 results in a relative activity of about 1.15 for cells treated with biotin-wortmannin and about 0.25 for cells treated with wortmannin. This shows a significant drop of ATM activity in cells treated with wortmannin but the activity of ATM gained from cells treated with biotin-wortmannin remains close to the control (DMSO treated) level. Therefore, this experiment provides further evidence that this compound can not effectively label proteins in intact cells.

Wortmannin itself and other biotin derivatives (Uptima, 2001) are hydrophobic enough to cross biological membranes and to enter into cells. The PEG-linker used was fairly hydrophilic which suggesting that the linker makes the whole compound too hydrophilic to cross membranes based on lipid bilayers. This linker was chosen to make the whole compound hydrophilic enough to be soluble in water for the use in biological systems.

### EXAMPLE 2

### Pull-Down Experiments as one Application of the Biotin Derivatives of Wortmannin

One application of the biotin-wortmannin compound that is based on the strong interaction of Biotin (vitamin H) and the tetrameric binding protein streptavidin or avidin, is to precipitate or pull-down members of the PI 3-kinase superfamily using streptavidin beads. An assay to pull-down ATM, ATR and DNA-PK, three members of the PIK-related kinase family, was successfully explored. The pull-down efficiency with ATR was usually around 80% at 50 μM biotin-wortmannin. The pull-down of the biotin-control did not result in any detectable amount of PIK-related kinases.

i) Optimizing of the Reaction Conditions

We were particularly interested in using the biotin-wortmannin compound to label members of the PIK-related kinases, namely ATM, ATR and DNA-PKcs. Because members of the PIK-related kinases family are primarily localized in the nucleus we used nuclear extract for our pull-down experiments.

Conditions for the pull-down were optimized by using different buffers, by varying the incubation time and temperature for the biotin-wortmannin incubation, and by varying the incubation time for the streptavidin pull-down. The use of different buffers had only a slight impact on the pull-down efficiency. Surprisingly, Tris-based buffered system seemed to be more effective than HEPES at pH 7.5. For efficient pull-down of ATR, incubation at 30° C. was necessary. However, for ATM and DNA-PK_{cs}, incubation on ice was sufficient to pull-down these kinases. Incubation times as little as 5 min had the same effect as incubation times up to 30 min. Freezing after incubation with biotin-wortmannin incubation also increased the pull-down efficiency. For the pull-down reaction with streptavidin-sepharose, incubation times ranging from between 5-30 min were tested but did not change the yield. Also, the denaturation/elution conditions from the streptavidin beads were optimized by using different dilutions of 6×SDS sample buffer and different incubation times at 95° C.

ii) Concentration Range to Determine Sensibility if Different PIK-Related Kinases Towards Biotin-Wortmannin (BW)

To determine which PIK-related kinases could be pulled-down with BW, we performed several affinity precipitations after incubation with BW concentrations ranging from 0.5-50 μM. As controls, we used the BW compound (negative) and nuclear extracts (positive). We probed a Western blot for the precipitation of ATR, ATM and DNA-PK_{cs} and received a pattern of different sensibility that is related to the IC₅₀s observed in vitro. DNA-PK_{cs} is the most sensitive member to precipitation with BW of the PIK-related kinases we tested and can be precipitated at concentrations as low as 0.5 μM. ATM also seems to be more sensitive to precipitation than ATR, and starts to give a signal at 2 μM. ATR is the least sensitive kinase to precipitation tested, and starts to pull-down at 10 μM.

iii) Specificity/Competition Reactions to Show Specificity

As we showed in the last experiment, different members of the PIK-related kinase family can be precipitated with biotin-wortmannin and we wanted to make sure that the pull-down reaction is based on a specific interaction between biotin-wortmannin and the PIK-related kinase. Therefore, we performed pull-down reactions with 30 μM biotin-wortmannin using HeLa nuclear extract. The samples used in the pull-down experiments were either untreated, or treated prior to the reaction with either 150 μM wortmannin, heating (95° C.) or 1 mM ATP. The proteins that were pull-down were analyzed via Western blot detecting DNA-PKcs, ATM and biotin-wortmannin.

Previous incubation with wortmannin significantly reduced the pull-down efficiency for ATM and DNA-PKcs, as only very little of these PIK-related kinases were detected. Previous incubation at 95° C. for denaturation prevented the pull-down reaction, as no ATM or DNA-PKcs were detected. Adding 1 mM ATP did not show an effect on the pull-down reaction.

The Western blot, labeled with streptavidin-HRP, showed that two high molecular weight bands (>250 kD), as well as a number of smaller protein, get competed out by wortmannin incubation or by heating. Although, there were also several bands that were not affected by the wortmannin incubation or heating.

Specificity

One concern was, considering the reduction of activity by adding the linker and biotin, we needed higher concentrations for our experiments and might also get in the unspecific range in which biotin-wortmannin could react with any amine. With the competition experiment, we showed that previous incubation with 5 fold excess wortmannin prevented almost all and previous denaturating of the proteins by heating prevented all precipitation of ATM and DNA-PKcs with the biotin-wortmannin (30 μM) compound. This shows that, at least at 30 μM BW, the reaction is specific as biotin-wortmannin seems to react at the same sites, a lysine in the active center at which wortmannin reacts (Wymann et al., 1996). As no ATM and DNA-PKcs could be detected after denaturation, it shows that the integrity of the catalytic center is necessary and the compound does not react with other amine residues outside the catalytic center.

iv) Combined Pull-Down Experiments for Biological Characterization of ATR

The ability to pull-down of members of the PIK-related kinase family offers the chance to also pull-down other proteins that specifically interact with these kinases. It is known, that ATR is found in different complexes in the cell, before and after damage. In addition, we have found that ATR also can bind to DNA. This interaction is indirect, and we have preliminary data that it is mediated by one or more proteins.

The BW compound of the invention can be used to isolate ATR-associated proteins. However, the IC₅₀ of biotin-wortmannin is relatively high for ATR. Therefore, several other proteins are also labeled with the BW reagent in extracts, which makes preparation of an ATR affinity reagent used with total cell extract difficult. Therefore, we tested the use of partially purified ATR for preparation of an affinity reagent. To obtain the affinity purified ATR, cells were transfected with Flag-tagged ATR, lysed and an anti-Flag immunoprecipitation was performed. Flag-tagged ATR was eluted with the Flag peptide. The same steps were carried out for untransfected cells to prepare a control extract/column. Eluted proteins were incubated with either 30 μM biotin-wortmannin or 30 μM biotin-control then isolated with streptavidin sepharose.

In the first two lanes, the proteins remaining on the Flag beads were shown. In the second lane (Flag-ATR transfected), compared to the untransfected lane, an extra band just above 250 kD is predominant which shows that a significant amount of ATR was not eluted from the beads. The last 4 lanes show the proteins than could be precipitated using either the biotin-wortmannin or biotin-control compound. For the identification of Flag-ATR associated proteins we compared the proteins that precipitated at 30 μM BW in the Flag-ATR transfected lane to the precipitated proteins with untransfected cells. This lane was further compared to the proteins that precipitated with the BC compound which are therefore precipitated by an unspecific interaction with the BC compound or the streptavidin resin. Considering all controls, 4 specific bands were detected between about 35 kD and 100 kD in the Flag-ATR BW pull-down-reaction.

In our lab, it has also been shown, that in vitro translated ATR, in contrast to cellular ATR, does not bind DNA, although the binding activity can be reconstituted by adding cellular lysate.

In a combined anti-Flag and biotin-wortmannin affinity approach, we were able to detect four specific Flag-ATR associated protein bands by silver stain among which might be the DNA binding activity.

### EXAMPLE 3

### Activity-Based Labeling Experiment

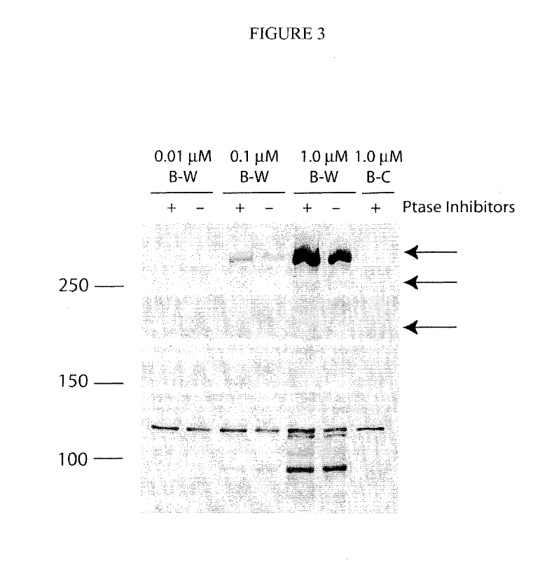

To determine if the biotin-wortmannin compound reacts with proteins in a manner that is dependent on their phosphorylation state, we have prepared lysates from confluent, human 293T cells in the presence or absence of a cocktail of phosphatase inhibitors (orthovanadate, sodium fluoride, beta-glycerophosphate, and microcystiene). These lysates were first precleared by mixing with streptavidin-resin, then incubated in the presence or absence of different doses of the biotin-wortmannin compound for 15 min at 4° C. An additional sample was incubated under the same conditions with a biotin-linker compound that does not possess wortmannin and therefore serves as a negative control for nonspecific binding. Lysates were then separated by SDS-PAGE and transferred to a PVDF membrane. The resulting blot was incubated with streptavidin-HRP and labeled proteins detected by enhanced chemiluminescence. As shown in the FIG. 3, several proteins were differentially labeled under the different lysis conditions. This suggests that different stimuli may lead to activation or inhibition of a given wortmannin-reactive kinase through phosphorylation or possibly other post-translational modifications and that this difference in activity could be detected with labeled wortmannin derivatives.

### EXAMPLE 4

### Fluorescently Labeled Wortmannin Derivatives

i) Preparation of BODIPY-FL Carboxylic Acid Derivatives

BODIPY-FL (hereafter called BODIPY) was purchased from Molecular Probes (D-2390, Eugene, Oreg.) and used without further purification. The BODIPY free amine (10.0 mg) was placed in a 10-mL round-bottom flask and dissolved in 2.0 mL dichloromethane. An additional 0.25 mL methanol was added to ensure solubility of the BODIPY starting material. Succinic anhydride was added in one portion (13 mg, 5 equivalents) and the clear reaction solution was allowed to stir overnight until the reaction was shown to be complete by thin layer chromatography analysis. The reaction mixture was concentrated to dryness under reduced pressure, and the contents of the flask were purified using silica gel chromatography (9:1 to 5:1 to 3:1 dichloromethane:methanol).

ii) Preparation of 11-O-deacetylwortmannin

The deacetylation of wortmannin was performed according to the procedure of Creemer et al. as described in their 1996 manuscript: “Synthesis and in vitro evaluation of new wortmannin esters: Potent inhibitors of phosphatidylinositol 3-kinase.” Creemer, L. C.; Kirst, H. A.; Vlahos, C. J.; Schultz, R. M. J. Med. Chem. 1996, 39, 5021-5024.

iii) Preparation of BODIPY-Wortmannin

The BODIPY carboxylic acid derivative (10.3 mg, 0.0237 mmol, 1.1 equivalents) was dissolved in 0.2 mL dichloromethane and treated with diisopropylcarbodiimide (0.0067 mL, 0.043 mmol, 2 equivalents) and 4-dimethylaminopyridine (3.1 mg, 0.026 mmol, 1.2 equivalents) and stirred at room temperature. A sample of 11-O-deacetylwortmannin (8.3 mg, 0.0215 mmol, 1.0 equivalents) dissolved in 0.1 mL dichloromethane was then added via syringe. Finally, a trace amount (<1 mg) of catalytic para-toluenesulfonic acid was added and the dark red solution was stirred overnight. At 24 h, TLC analysis showed that the reaction was incomplete, so an additional aliquot of diisopropylcarbodiimide (0.015 mL, 4 equivalents) was added. The reaction was complete at 66 h, and the red solution was transferred to a 10-mL conical flask, concentrated to dryness under reduced pressure, and purified using silica gel chromatography (0% methanol to 2% methanol to 5% methanol in ethyl acetate). A sample of BODIPY-wortmannin was isolated (6.0 mg, 0.0075 mmol) and characterized using NMR spectroscopy and mass spectrometry.

### REFERENCES

Abraham, R. T. (2001). “Cell cycle checkpoint signalling through the ATM and ATR kinaes,” Genes Dev 15, 2177-96.

Anderson, C. W., and Lees-Miller, S. P. (1992). “The nuclear serine/threonine protein kinase DNA-PK,” Crit Rev Eukaryot Gene Expr 2, 283-314.

Arcaro, A., and Wymann, M. P. (1993). “Wortmannin is a potent phosphatidylinositol 3kinase inhibitor; the role of phosphatidylinositol 3,4,5-trisphosphate in neutrophil responses,” Biochem J 296, 297-301.

Baggiolini, M., Dewald, B., Schnyder, J., Ruch, W., Cooper, P. H., and Payne, T. G. (1987). “Inhibition of the phagocytosis-induced respiratory burst by the fungal metabolite wortmannin and some analogues,” Exp Cell Res 169, 408-18.

Barlow, C., Hirotsune, S., Paylor, R., Liyanage, M., Lckhaus, M., Collins, F., Shiloh, Y., Crawley, J. N., Ried, T., Tagle, D., and Wynshaw-Boris, A. (1996). “Atm-deficient mice: a paradigm of ataxia telangiectasia,” Cell 86, 159-71.

Bentley, N. J., Holtzman, D. A., Flaggs, G., Keegan, K. S., DeMaggio, A., Ford, J. C., Hookstra, M., and Carr, A. M. (1996). “The Schizosaccharomyces pombe rad3 checkpoint gene,” Embo J 15, 6641-51.

Bochar, D. A., Wang, L., Beniya, H., Kinev, A., Xue, Y., Lane, W. S., Wang, W., diisopropylcarbodiimide (0.0067 mL, 0.043 mmol, 2 equivalents) and 4-dimethylaminopyridine (3.1 mg, 0.026 mmol, 1.2 equivalents) and stirred at room temperature. A sample of 11-O-deacetylwortmannin (8.3 mg, 0.0215 mmol, 1.0 equivalents) dissolved in 0.1 mL dichloromethane was then added via syringe. Finally, a trace amount (<1 mg) of catalytic para-toluenesulfonic acid was added and the dark red solution was stirred overnight. At 24 h, TLC analysis showed that the reaction was incomplete, so an additional aliquot of diisopropylcarbodiimide (0.015 mL, 4 equivalents) was added. The reaction was complete at 66 h, and the red solution was transferred to a 10-mL conical flask, concentrated to dryness under reduced pressure, and purified using silica gel chromatography (0% methanol to 2% methanol to 5% methanol in ethyl acetate). A sample of BODIPY-wortmannin was isolated (6.0 mg, 0.0075 mmol) and characterized using NMR spectroscopy and mass spectrometry.

### REFERENCES

Abraham, R. T. (2001). “Cell cycle checkpoint signalling through the ATM and ATR kinaes,” Genes Dev 15, 2177-96.

Anderson, C. W., and Lees-Miller, S. P. (1992). “The nuclear serine/threonine protein kinase DNA-PK,” Crit Rev Eukaryot Gene Expr 2, 283-314.

Arcaro, A., and Wymann, M. P. (1993). “Wortmannin is a potent phosphatidylinositol 3kinase inhibitor; the role of phosphatidylinositol 3,4,5-trisphosphate in neutrophil responses,” Biochem J296, 297-301.

Baggiolini, M., Dewald, B., Schnyder, J., Ruch, W., Cooper, P. H., and Payne, T. G. (1987). “Inhibition of the phagocytosis-induced respiratory burst by the fungal metabolite wortmannin and some analogues,” Exp Cell Res 169, 408-18.

Barlow, C., Hirotsune, S., Paylor, R., Liyanage, M., Lckhaus, M., Collins, F., Shiloh, Y., Crawley, J. N., Ried, T., Tagle, D., and Wynshaw-Boris, A. (1996). “Atm-deficient mice: a paradigm of ataxia telangiectasia,” Cell 86, 159-71.

Bentley, N. J., Holtzman, D. A., Flaggs, G., Keegan, K. S., DeMaggio, A., Ford, J. C., Hookstra, M., and Carr, A. M. (1996). “The Schizosaccharomyces pombe rad3 checkpoint gene,” Embo J 15, 6641-51.

Bochar, D. A., Wang, L., Beniya, H., Kinev, A., Xue, Y., Lane, W. S., Wang, W.,

Kashanchi, F., and Shiekhattar, R. (2000), BRCA1 is associated with a human SWI/SNF-related complex; linking chromatin remodeling to breast cancer, Cell 102, 25765.

Bogyo, M., McMaster, J. S., Gaczynska, M., Tortorella, D., Goldberg, A. L., and Ploegh, H. (1997). “Covalent modification of the active site threonine of protmomal beta subunits and the Escherichia coli homolog Hs1V by a new class of inhibitors,” Proc Natl Acad Sci USA 94,6629-34.

Bosma, M. J., and Carroll, A. M. (1991). “The SCID mouse mutant: definition, characterization, and potential uses,” Annu Rev Immunol 9, 323-50.

Bosotti, R., Isacehi, A., and Sounhammer, E. L. (2000). “FAT: a novel domain in PIK-related kinases,” Trends Biochem Sci 25, 225-7.

Brian, P. W., Curtis, P. J., H. G., H., and G. L. F., N. (1957). “Wortmannin, an antibiotic produced by Penicillium Wortmanni,” Trans Br Mycol Soc 40, 365-368.

Brown, E. J., Albers, M. W., Shin, T. B., Ichikawa, K., Keith, C. T., Lane, W. S., and Schreiber, S. L. (1994). “A mammalian protein targeted by G1-arresting rapamycinreceptor complex,” Nature 369, 756-8.

Brown, E. J., and Baltimore, D. (2000). “ATR disruption leads to chromosomal fragmentation and early embryonic lethality,” Genes Dev 14, 397-402.

Brunn, G. J., Williams, J., Sabers, C., Wiederrecht, G., Lawrence, J. C., Jr., and Abraham, R. T. (1996). “Direct inhibition of the signaling functions of the mammalian target of rapamycin by the phosphoinositide 3-kinase inhibitors, wortmannin and LY294002,” Embo J 15, 5256-67.

Chiu, M. I., Katz, H., and Berlin, V. (1994). “RAPTI, a mammalian homolog of yeast Tor, interacts with the FKBP121rapamycin complex,” Proc Natl Acad Sci USA 91, 12574-8.

Cimprich, K. A., Shin, T. S., Keith, C. T., and Schreiber, S. L. (1996), “cDNA cloning and gene mapping of a candidate human cell cycle checkpoint protein,” Proc Nall Acad Sci USA 93,2850-5.

Cliby, W. A., Roberts, C. J., Cimprich, K. A., Stringer, C. M., Lamb, J. R, Schreiber, S. L., and Friend, S. H. (1998). “Overexpression of a kinase-inactive ATR protein causes sensitivity to DNA-damaging agents and defects in cell cycle checkpoints,” Embo J 17, 159-69.

Cortez et al. (2001). “ATR and ATRIP: partners in development in checkpoint signaling,” Science 294:1713-6.

Cross, M. J., Stewart, A., Hodgkin, M. N., Kerr, D. J., and Wakelam, M. J. (1995). “Wortmannin and its structural analogue demethoxyviridin inhibit stimulated phospholipase A2 activity in Swiss 3T3 cells. Wortmannin is not a specific inhibitor of phosphatidylinositol 3-kinase,” J Biol Chem 270, 25352-5.

Davies, S. P., Reddy, If., Caivano, M., and Cohen, P. (2000). “Specificity and mechanism of action of some commonly used protein kinase inhibitors,” Biochem J 351, 95-105.

de Klein, A., Muijtjens, M., van Os, R., Verhoeven, Y., Smit, B., Carr, A. M., Lehmann, A. R., and Hoeijmakers, J. H. (2000). “Targeted disruption of the cell-cycle checkpoint gene ATR leads to early embryonic lethality in mice,” Curr Biol 10, 479-82.

Denning, G., Famieson, L., Maquat, L. E., Thompson, E. A., and Fields, A. P. (2001). “Cloning of a novel phosphatidylinositol kinase-related kinase; characterization of the human SMG-1 RNA surveillance protein,” J Biol Chem 276, 22709-14.

Duroeher, D., and Jackson, S. P. (2001). “DNA-PK, ATM and ATR as sensors of DNA damage: variations on a theme?,” Curr Opin Cell Biol 13, 225-31.

Gottlieb, T. M., and Jackson, S. P. (1993). The DNA-dependent protein kinase: requirement for DNA ends and association with Ku antigen, Cell 72, 131-42.

Grant, P. A., Schieltz, D., Pray-Grant, M. G., Yates, J. R., 3rd, and Workman, J. L. (1998), “The ATM-related cofactor Tral is a component of the purified SAGA complex,” Mol Cell 2, 863-7.

Greenbaum, D., Medzihradszky, K. F., Burlingame, A., and Rogyo, M. (2000). “Epoxide electrophiles as activity-dependent cysteine protease profiling and discovery tools,” Chem Biol 7, 569-81.

Hartley, K. U., Gell, D., Smith, G. C., Zhang, H., Diveehn, N., Connelly, M. A., Admon, A., Lm-Miller, S. P., Anderson, C. W., and Jackson, S. P. (1995). “DNA dependent protein kinase catalytic subunit: a relative of phosphatidylinositol 3-kinase and the ataxia telangiectasia gene product,” Cell 82, 849-56.

Izzard, R. A., Jackson, S. P., and Smith, G. C. (1999). “Competitive and noncompetitive inhibition of the DNA-dependent protein kinase,” Cancer Res 59, 2581-6.

Kanaar, 1″d., Hoeijmakers, J. H., and van Gent, D.C. (1998). “Molecular mechanisms of DNA double strand break repair,” Trends Cell Biol 8, 483-9.

Keith, C. T., and Schreiber, S. L. (1995). “PIK-related kinases: DNA repair, recombination, and cell cycle checkpoints,” Science 270, 50-1.

Kidd, D., Liu, Y., and Cravatt, B. F. (2001). “Profiling serine hydrolase activities in complex proteomes,” Biochemistry 40,4005-15.

Knipe, D. M., Fields, B. N., Howley, P. M., and Griffin, D. E. (2001). “Fields' virology, 4th ed edn” (Philadelphia, Lippincott Williams & Wilkins).

Kuruvilla, F. G., and Schreiber, S. L. (1999), “The PIK-related kinases intercept conventional signaling pathways,” Chem Biol 6, R129-36.

Lees-Miller, S. P., Sakeguchi, X., U11 rieh, S. J., Appella, F., and Anderson, C. W. (1992). “Human DNA-activated protein kinase phosphorylates serines 15 and 37 in the amino-terminal transactivation domain of human p53,” Mol Cell Biol 12, 50419.

Liu, Q., Guntuku, S., Cui, X. S., Matsuoka, S., Cortez, D., Tamai, K., Lao, G., Carattini-Rivera, S., DeMayo, F., Bradley, A., Donehower, L. A., and Elledge, S. J. (2000). “Chk1 is an essential kinase that is regulated by Atr and required for the G(2)/M DNA damage checkpoint,” Genes Dev 14,1448-59.

Liu, Y., Patricelli, M. P., and Cravatt, B. F. (1999). “Activity-based protein profiling; the serine hydrolases,” Proc Natl Acad Sci USA 96, 14694-9.

Lukashok, S. A., and Horwitz, M. S. (1998). “New perspectives in adenoviruses,” Curr Clin Top Infect Dis 18, 286-305.

Meyn, M. S. (1995), “Ataxia-telanglectasia and cellular responses to DNA damage,” Cancer Res 55, 5991-6001.

Park J., Kunjibettu, S., McMahon, S. B., and Cole, M. D. (2001), “The ATM-related domain of TRRAP is required for histone acetyltransferase recruitment and Myc-dependent oncogenesis,” Genes Dev 15, 1619-24.

Powis, G., Bonjouklian, R., Berggren, M. M., Gallegos, A., Abraham, R., Ashendel, C., Zalkow, L., Matter, W. F., Dodge, J., Grindey, G., and et al. (1994). “Wortmannin, a potent and selective inhibitor of phosphatidylinositol-3-kinase,” Cancer Res 54, 241923.

Sabers, C. J., Martin, M. M., Brunn, G. J., Williams, J. M., Dumont, F. J., Wiederrecht, G., and Abraham, R. T. (1995), “Isolation of a protein target of the FIOP12-rapamycin complex in mammalian cells,” J Biol Chem 270, 815-22.

Sarkaria, J. N., Tibbetts, R. S., Busby, E. C., Kennedy, A. P., Hill, D. E., and Abraham, R T. (1998). “Inhibition of phosphoinositide 3-kinase related kinases by the radiosensitizing agent wortmannin,” Cancer Res 58, 4375-82,

Schmidt, D. R., and Schreiber, S. L. (1999). “Molecular association between ATR and two components of the nucleosome remodeling and deacetylating complex, HDAC2 and CHD4,” Biochem 39, 14711-14717.

Shiloh, Y. (2001). “ATM and ATR: networking cellular responses to DNA damage,” Curr Opin Genet Dev 11, 71-7.

Smith, G. C., Cary, R. B., Lakin, N. D., Hann, B. C., Teo, S. H., Chen, D. J., and Jackson, S. P. (1999a), “Purification and DNA binding properties of the ataxia-telangiectasia gene product ATM,” Proc Natl Acad Sci USA 96,11134-9.

Smith, G. C., and Jackson, S. P. (1999b). “The DNA-dependent protein kinase,” Genes Dev 13, 916-34.

Stein, R. C., and Waterfield, M. D. (2000). “P13-kinase inhibition; a target for drug development?,” Mol Med Today 6, 347-57.

Suzuki, K., Codama, S., and Watanabe, M. (1999), “Recruitment of ATM protein to double strand DNA irradiated with ionizing radiation,” J Biol Chem 274, 25571-5.

Tibbetts, R. S., Brumbaugh, K. M., Williams, J. M., Sarharia, J. N., Cliby, W. A., Shieh, S. Y., Taya, Y., Prives, C., and Abraham, R. T. (1999). “A role for ATR in the DNA damage-induced phosphorylation of p53,” Genes Dev 13,152-7.

Tibbetts, R. S., Cortez, D., Brumbaugh, K. M., Scully, R., Livingston, D., Elledge, S. J., and Abraham, R. T. (2000), “Functional interactions between BRCA1 and the checkpoint kinase ATR during genotoxic stress,” Genes Dev 14, 2989-3002.

Ui, M., Okada, T., Hazeki, K., and Hazeld, O. (1995). “Wortmanuin as a unique probe for an intracellular signaling protein, phosphoinositide 3-kinase,” Trends Biochem Sci 20, 303-7.

Uptima. (2001). Product catalog,

Vlahos, C. J., Matter, W. F., Hui, K. Y., and Brown, R. F. (1994). “A specific inhibitor of phosphatidylinositol 3-kinase, 2-(4-morpholinyl)-S-phenyl-4H-1-benzopyran-4-one (LY294002),” J Biol Chem 269, 5241-5.

Walker, F. H., Pacold, M. F., Perisic, O., Stephens, L., Hawkins, P. T., Wymann, M. P., and Williams, R. L. (2000). “Structural determinants of phosphoinositide 3-kinase inhibition by wortmannin, LY294002, quercetin, myricetin, and staurosporine,” Mol Cell 6,909-19.

Wang, Y., Cortez, D., Yazdi, P., Neff, N., Flledge, S. J., and Qin, J. (2000). “BASC, a super complex of BRCA1-associated proteins involved in the recognition and repair of aberrant DNA structures,” Genes Dev 14, 927-39.

Woscholski, M, Kodaki, T., McKinnon, M., Waterfield, M. D., and Parker, P. J. (1994). “A comparison of demethoxyviridin and wortmannin as inhibitors of phosphatidylinositol 3-kinase,” FEES Lett 342, 109-14.

Wright, J. A., Keegan, X. S., Herendeen, D. R., Bentley, N. J., Carr, A. M., Hoekstra, M. F., and Concannon, P. (1998). “Protein kinase mutants of human ATR increase sensitivity to UV and ionizing radiation and abrogate cell cycle checkpoint control,” Proc Nad Acad Sci USA 95, 7445-50.

1. A method for identifying binding partners which bind to a wortmannin moiety comprising:
a. providing a wortmannin bait moiety including wortmannin or an analog thereof being derivatized to a solid support or molecular or chemical tag for purifying or identifying the bait moiety;
b. contacting the bait moiety with a library of binding partners and isolating from the library binding partners, if any, which bind to the bait moiety;
c. identifying those members of the binding partner library which specifically bind to the bait moiety.

2. A method for identifying kinases comprising:
a. providing a lipid kinase inhibitor bait moiety being derivatized to a solid support or molecular or chemical tag for purifying or identifying the bait moiety, which lipid kinase inhibitor forms a covalent adduct with lipid kinases and has a Ki for inhibition of a lipid kinase of 50 μM or less;
b. contacting the bait moiety with a library of binding partners and isolating from the library binding partners, if any, which bind to the bait moiety;
c. identifying those members of the binding partner library which specifically bind to the bait moiety.

3. The method of claim 1 or 2, wherein the bait moiety is a covalent inhibitor of a phosphatidylinositol kinase.

4. The method of claim 2, wherein the lipid kinase inhibitor is selected from the group consisting of wortmannin, hydroxywortmannin, LY294002, demethoxyviridin, quercetin, myricetin and staurosporine, and analogs thereof.

5. The method of claim 1, wherein the wortmannin or analog thereof is derivatized to the solid support or molecular or chemical tag through a cross-linking moiety which is covalently attached to C11 of the wortmannin or wortmannin analog.

6. The method of claim 1, wherein the bait moiety is represented in the general formula 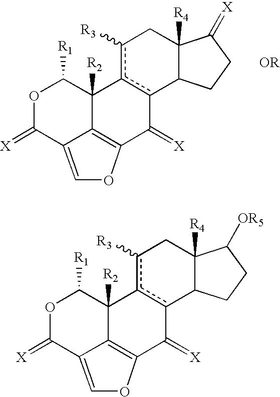
wherein
X, independently for each occurrence, represents O or S,
R₁ represents —(CH₂)ₙ—X—R₅,
R₂, R₄ and R₅, independently, represent H or a C1-C6 alkyl,
R₃ represents S-L-,
S represents a solid support or molecular or chemical tag for purifying or identifying the bait moiety,
L represents a linker, and
n is 0, 1, 2 or 3.

7. The method of any of claims 1 or 2, wherein the library of binding partners is a polypeptide library.

8. The method of claim 7, wherein the polypeptide library is an expression library.

9. The method of claim 8, wherein the polypeptide library is derived from replicable genetic display packages.

10. The method of claim 7, wherein the polypeptide library is a cell lysate or partially purified protein preparation.

11. The method of any of claims 1 or 2, wherein the identity of those members of the binding partner library which specifically bind to the bait moiety is determined by mass spectroscopy.

12. A drug screening assay comprising:
a. providing a reaction mixture including a binding partner identified by the method of claim 1 or 2;
b. contacting the binding partner with a test compound;
c. determining if the test compound specifically binds to the binding partner.

13. The method of claim 12, wherein the test compound which is identified as able to bind to the binding partner is further tested for the ability to inhibit or activate one or more cellular kinases.

14. The method of claim 12, wherein the reaction mixture is a whole cell.

15. The method of claim 12, wherein the reaction mixture is a cell lysate or purified protein composition.

16. A method of conducting a drug discovery business comprising:
a. providing a wortmannin bait moiety being derivatized to a solid support or molecular or chemical tag for purifying or identifying the bait moiety;
b. contacting the wortmannin bait moiety with a library of binding partners;
c. identifying those members of the binding partner library which specifically bind to the wortmannin bait moiety;
d. providing a reaction mixture including a binding partner identified in step (c) as able to specifically bind to the wortmannin bait moiety;
e. contacting the binding partner with a test compound;
f. determining if the test compound specifically binds to the binding partner;
g. formulating a pharmaceutical preparation including one or more compounds identified in step (f) as able to inhibit or mimic the activity of a wortmannin moiety.

17. A method of conducting a drug discovery business comprising:
a. identifying those members of a binding partner library which specifically bind to a wortmannin bait moiety;
b. identifying compounds by their ability to agonize or antagonize a binding partner identified in step (a);
c. conducting therapeutic profiling of a compound identified in step (b), or further analogs thereof, for efficacy and toxicity in animals;
d. formulating a pharmaceutical preparation including one or more agents identified in step (iii) as having an acceptable therapeutic profile.

18. The method of claim 17, including an additional step of establishing a distribution system for distributing the pharmaceutical preparation for sale, and/or establishing a sales group for marketing the pharmaceutical preparation.

19. A method of conducting a target discovery business comprising:
a. providing a wortmannin bait moiety being derivatized to a solid support or molecular or chemical tag for purifying or identifying the bait moiety;
b. contacting the wortmannin bait moiety with a library of binding partners;
c. identifying those members of the binding partner library which specifically bind to the wortmannin bait moiety;
d. licensing, to a third party, the rights for drug development for a binding partner identified in step (c) as able to specifically bind to the wortmannin bait moiety.

20. A method of generating a pharmaceutical preparation including one or more compounds capable of binding to a binding partner binding to a wortmannin bait moiety, comprising:
a. providing a wortmannin bait moiety being derivatized to a solid support or molecular or chemical tag for purifying or identifying the bait moiety;
b. contacting the wortmannin bait moiety with a library of binding partners;
c. identifying those members of the binding partner library which specifically bind to the wortmannin bait moiety;
d. providing a reaction mixture including a binding partner identified in step (c) as able to specifically bind to the wortmannin bait moiety;
e. contacting the binding partner with a test compound;
f. determining if the test compound binds to the binding partner;
g. formulating a pharmaceutical preparation including one or more compounds identified in step (f) as able to inhibit or mimic the activity of a wortmannin moiety.

21. A composition including a bait moiety represented in the general formula 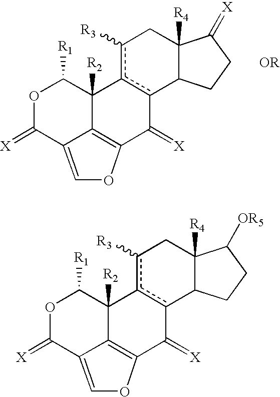
wherein:
X, independently for each occurrence, represents O or S,
R₁ represents —(CH₂)ₙ—X—R₅,
R₂, R₄ and R₅, independently, represent H or a C1-C6 alkyl,
R₃ represents S-L-,
S represents a solid support or molecular or chemical tag for purifying or identifying the bait moiety,
L represents a linker, and
n is 0, 1, 2 or 3.

22. A method for profiling wortmannin-binding components of a cellular lysate, comprising:
a. providing a wortmannin bait moiety including wortmannin or an analog thereof being derivatized to a solid support or molecular or chemical tag for purifying the bait moiety;
b. contacting the bait moiety with a cell lysate and isolating from the lysate binding partners, if any, which bind to the bait moiety;
C. identifying those binding partners which specifically bind to the bait moiety.

23. The method of claim 21, where the identity of the binding partners which specifically bind to the bait moiety are determined by mass spectroscopy.

24. A method for profiling wortmannin-binding components of a cell, comprising:
a. providing a wortmannin bait moiety including wortmannin or an analog thereof being derivatized to a molecular or chemical tag for visualizing the bait moiety in a cell;
b. contacting the bait moiety with a cell and determining the cellular localization(s) of the bait moiety.

25. A method for profiling wortmannin-binding components, comprising:
a. providing a wortmannin bait moiety including wortmannin or an analog thereof being derivatized to a molecular or chemical tag for visualizing the bait moiety in a cell;
b. contacting the bait moiety with a library of binding partners;
d. separating the components using SDS-PAGE and determining the location of the bait moiety on the gel.

26. A method for profiling wortmannin-binding components comprising:
a. providing a wortmannin bait moiety including wortmannin or an analog thereof being derivatized to a molecular or chemical tag for purifying or identifying the bait moiety;
b. contacting the bait moiety with a library of cellular components, wherein the library of cellular components has been derived from cells that have been exposed to a first set of conditions, and identifying those members of the cellular component library which specifically bind to the bait moiety;
c. contacting the bait moiety with a library of cellular components, wherein the library of cellular components has been derived from cells that have been exposed to a second set of conditions, and identifying those members of the cellular component library which specifically bind to the bait moiety;
d. comparing the cellular components identified in (b), with the cellular components identified in (c), wherein a difference between the cell components identified in (b) and the cellular components identified in the (c) indicates that the difference in the conditions cause a change in the cellular component that can be used to profile the binding components.

27. A method for identifying the phosphorylation state of wortmannin binding partners comprising:
a. providing a wortmannin bait moiety including wortmannin or an analog thereof being derivatized to a molecular or chemical tag for purifying or identifying the bait moiety;
b. contacting the bait moiety with a library of cellular components that have been derived from cells that have been exposed to a first set of conditions, and identifying those members of the cellular component library which specifically bind to the bait moiety;
c. contacting the bait moiety with a library of cellular components that have been derived from cells that have been exposed to a second set of conditions, and identifying those members of the cellular component library which specifically bind to the bait moiety;
d. comparing the cellular components identified in (b), with the cellular components identified in (c), thereby identifying cellular components that differently phosphorylated between the first and second set of conditions.

28. The method of claim 26 or 27, wherein the first set of conditions is the presence of phosphatase inhibitors, and the second set of conditions is the absence of phosphatase inhibitors.

29. The method of claim 26 or 27, wherein the first set of conditions is the presence of one or more growth factors, and the second set of conditions is the absence of said one or more growth factors.

30. A method for identifying the phosphorylation state of wortmannin binding partners comprising:
a. providing a wortmannin bait moiety including wortmannin or an analog thereof being derivatized to a molecular or chemical tag for purifying or identifying the bait moiety;
b. contacting the bait moiety with a library of cellular components that have been derived from differentiated cells, and identifying those members of the cellular component library which specifically bind to the bait moiety;
c. contacting the bait moiety with a library of cellular components that have been derived from undifferentiated cells, and identifying those members of the cellular component library which specifically bind to the bait moiety;
d. comparing the cellular components identified in (b), with the cellular components identified in (c), thereby identifying cellular components that are differently phosphorylated between differentiated and undifferentiated cells.

31. A method for identifying the phosphorylation state of wortmannin binding partners comprising:
a. providing a wortmannin bait moiety including wortmannin or an analog thereof being derivatized to a molecular or chemical tag for purifying or identifying the bait moiety;
b. contacting the bait moiety with a library of cellular components that have been derived from cancer cells, and identifying those members of the cellular component library which specifically bind to the bait moiety;
c. contacting the bait moiety with a library of cellular components that have been derived from non-cancer cells, and identifying those members of the cellular component library which specifically bind to the bait moiety;
d. comparing the cellular components identified in (b), with the cellular components identified in (c), thereby identifying cellular components that are differently phosphorylated between cancer and non-cancer cells.

32. The method of claim 26, 27, 30 or 31, wherein the members of the cellular components that specifically bind to the bait moiety are identified by SDS-PAGE.

33. The method of claim 26, 27, 30 or 31, wherein the members of the cellular components that specifically bind to the bait moiety are affinity enriched.

34. The method of claim 26, 27, 30 or 31, wherein the members of the cellular components are identified using mass spectroscopy.
